(19) United States
(12) Patent Application Publication
(10) Pub. No.: US 2026/0201369 A1
(43) Pub. Date: Jul. 16, 2026

(54) CIRCRNA EXPRESSION CONSTRUCT

(71) Applicant: CIRCIO AB, Hässelby (SE)

(72) Inventors: Thomas HANSEN, Hässelby (SE); Eoghan O'LEARY, Hässelby (SE)

(21) Appl. No.: 19/136,063

(22) PCT Filed: Dec. 7, 2023
(86) PCT No.: PCT/EP2023/084788

§ 371 Date: Jun. 5, 2025

(30) Foreign Application Priority Data
Dec. 7, 2022 (EP) 22212051.1

(51) Int. Cl. C12N 15/11; A61K 31/7105; A61K 38/16; A61K 48/00; C12N 9/12; C12N 15/63

(52) U.S. Cl. CPC C12N 15/11; A61K 31/7105; A61K 38/16; A61K 48/0066; C12N 9/1247; C12N 15/63; C12N 2830/34; C12N 2840/206; C12N 2840/44; C12Y 207/07006

(57) ABSTRACT

The present invention relates to a nucleic acid molecule encoding a circular RNA (circRNA). In another aspect the invention relates to a circRNA encoded by the nucleic acid and a vector and host cell comprising the nucleic acid. Further includes are methods of producing the circRNA.

The present invention relates to a nucleic acid molecule encoding a circular RNA (circRNA). In another aspect the invention relates to a circRNA encoded by the nucleic acid and a vector and host cell comprising the nucleic acid. Further includes are methods of producing the circRNA.

### BACKGROUND OF THE INVENTION

Circular RNA (circRNA) constitutes a novel class of long non-coding RNAs characterized as covalently closed molecules. CircRNAs are typically produced by a non-linear ‘backsplicing’ event using a downstream splice donor (SD) and an upstream splice acceptor (SA) in contrast to conventional linear splicing. Endogenous circular RNA has been studied intensively in the past decade, and while some controversies on functional relevance exists, it is widely appreciated that circRNAs by virtue of their circular nature are resistant towards exonucleolytic decay, and therefore circRNAs comprise a very stable class of RNA with half-lives greatly exceeding that of conventional linear mRNA.

During the last decade knowledge of circRNA production, both in vitro and in vivo, has improved significantly, and consequently, the therapeutic potential of a durable circular RNA concept with engineered functionalities has now emerged. In broad terms, circRNA production can be achieved by two distinct mechanisms, either 1) using group-I-intron derived ribozymatic circularization shown effective for in vitro production, or 2) using spliceosome-based backsplicing similar to endogenous circRNA biogenesis useful for in vivo production. In the latter setup, inserting flanking inverted elements is known to stimulate backsplicing dramatically, proposedly by positioning the splice sites involved in close proximity. While circRNAs are devoid of 5′cap and 3′ polyA tails and thus per se not substrates for translation, the insertion of IRES (internal ribosome entry sites) effectively converts non-coding circRNAs into highly effective protein-encoding molecules. Thus, the therapeutic use of circular RNA as templates for durable protein production has garnered significant attention.

The present invention provides an improved circRNA expression cassette to facilitate a high level of stable circRNA expression in vivo. Surprisingly, it was found that IRES positioning within a circRNA expression cassette had high relevance for circRNA yield.

### SUMMARY OF THE INVENTION

A nucleic acid encoding a circular RNA (circRNA) is disclosed herein, wherein the IRES driving the translation of the circRNA is positioned in the specified position with regard to the splice sites required for backsplicing of the circRNA resulting in a superior backsplicing efficiency.

In a first aspect, the present invention provides a nucleic acid molecule encoding a circular RNA (circRNA), wherein the nucleic acid molecule comprises:
A) an expression cassette comprising:
   (i) a circRNA expression cassette comprising:
      (a) a nucleic acid comprising or consisting of a continuous or split open reading frame (ORF) encoding at least one protein, and
      (b) an internal ribosome entry site (IRES) operably linked to the ORF to direct translation of the ORF;
   (ii) a first backsplicing site that is positioned 5′ of the IRES; and
   (iii) a second backsplicing site that is positioned 3′ of the IRES; and optionally
B) a first inverted repeat (IR) element positioned 5′ of the first backsplicing site and a second IR element positioned 3′ of the second backsplicing site; and/or a promoter operably linked to the expression cassette to direct expression of the expression cassette;
wherein the IRES is positioned within the circRNA expression cassette in that:
(I) the 5′ end of the IRES and the 3′ end of the first backsplicing site are separated by at least 50 nucleotides, preferably by at least 200 more preferably by at least 300 nucleotides, and
(II) the 3′ end of the IRES and the 5′ end of the second backsplicing site are separated by at least 300 nucleotides, preferably by at least 350 nucleotides.

### LIST OF FIGURES

In the following, the content of the figures comprised in this specification is described. In this context please also refer to the detailed description of the invention above and/or below.

FIG. 1: refers to (A) schematic of continuous ORF (upper) and split ORF (lower) designs. Depicted are the relative position of promoter, inverted repeats (IR), splice acceptor (SA), IRES, open reading frame (ORF) and splice donor (SD). (B) Western blot of ICOS-L protein expression from A375 cells transfected with either empty vector control (EV), or circICOSL expressing plasmids in both continuous and split ORF design containing either CVB3 or EMCV IRES. j-actin used as loading control. (C) RT-qPCR quantification of circICOSL expression relative to GAPDH mRNA from A375 cells transfected with circICOSL expressing plasmids in both continuous and split ORF design containing either CVB3 or EMCV IRES. Data for three biological replicates are shown. (D) Agarose gel depicting RT-PCR amplification of circICOSL from A375 cells transfected with either empty vector control (EV), or circICOSL expressing plasmids in both continuous and split ORF design containing either CVB3 or EMCV IRES.

FIG. 2: refers to (A) schematic of eGFP split ORF designs. CircRNA schematic depicts relative position of IRES within split eGFP ORF. (B) Agarose gel depicting RT-PCR amplification of circEGFP from A375 cells transfected with eGFP_FLAG_Split_ORF circRNA plasmids. (C) RT-qPCR quantification of circEGFP expression relative to GAPDH mRNA from A375 cells transfected with eGFP_FLAG_Split_ORF circRNA plasmids. Data for two biological replicates are shown. (D) Western blotting analysis of A375 cells transfected with eGFP_FLAG Split_ORF circRNA plasmids, probed with the denoted antibodies FLAG, GFP and β-actin (loading control).

FIG. 3: refers to (A) schematic of eGFP_FLAG_Split_IRES circRNA designs. CircRNA schematic depicts relative position of split_IRES and eGFP ORF. (B) Agarose gel depicting RT-PCR amplification of circEGFP from A375 cells transfected with eGFP_FLAG_Split_IRES circRNA plasmids. (C) RT-qPCR quantification of circEGFP expression relative to GAPDH mRNA from A375 cells transfected with eGFP_FLAG_Split_IRES circRNA plasmids. Data for two biological replicates are shown.

FIG. 4: refers to (A) schematic of eGFP_HIPK3_spacer circRNA designs. CircRNA schematic depicts relative position of HIPK3_spacer (black), IRES (light grey) and eGFP ORF (dark grey). (B) Agarose gel depicting RT-PCR amplification of circEGFP from A375 cells transfected with eGFP_FLAG_HIPK3_spacer circRNA plasmids. (C) RT-qPCR quantification of circEGFP expression relative to GAPDH mRNA from A375 cells transfected with eGFP_FLAG_HIPK3_spacer circRNA plasmids. Data for two biological replicates are shown.

FIG. 5: refers to (A) Schematic of mRNA and circRNA designs. CircRNA schematic depicts relative position of IRES and transgene ORF. (B-D) Western blotting analysis of A375 cells transfected with mRNA and circRNA plasmids encoding ADA_FLAG (B), ICOSL (C) and Renilla (D), probed with the denoted antibodies FLAG, ICOSL, Renilla and β-actin (loading control). (E-G) RT-aPCR quantification of circADA (E), circICOSL (F) and circRenilla (G) expression relative to GAPDH mRNA from A375 cells transfected with mRNA and circRNA plasmids encoding ADA_FLAG, ICOSL and Renilla. Data for two biological replicates are shown.

FIG. 6: refers to (A) Western blotting analysis of A375 cells transfected with ICOSL_Split_CVB3 circRNA plasmids, probed with the denoted antibodies ICOSL and 0-actin (loading control). Schematic ofICOSL_Split_CVB3 circRNA expression cassette designs depicting relative position of CVB3 IRES (dark grey) and ICOSL ORF (light grey). (B) RT-qPCR quantification of ICOSL expression relative to GAPDH mRNA from A375 cells transfected with ICOSL_Split_CVB3 circRNA plasmids. Data for two biological replicates are shown.

FIG. 7: refers to (A) Western blotting analysis of A375 cells transfected with eGFP_Split_CVB3 circRNA plasmids probed with the denoted antibodies GFP and β-actin (loading control). Schematic of eGFP_Split_CVB3 circRNA expression cassette designs, depicting relative position of CVB3 IRES (dark grey) and eGFP ORF (light grey). (B) RT-qPCR quantification of eGFP expression relative to GAPDH mRNA from A375 cells transfected with eGFP_Split_CVB3 circRNA plasmids. Data for two biological replicates are shown. (C) Western blotting analysis of A375 cells transfected with eGFP_Split_EMCV_circRNA plasmids probed with the denoted antibodies GFP and β-actin (loading control). Schematic of eGFP_Split_EMCV circRNA expression cassette designs. CircRNA schematic depicts relative position of EMCV IRES (dark grey) and eGFP ORF (light grey). (D) RT-qPCR quantification of eGFP expression relative to GAPDH mRNA from A375 cells transfected with eGFP_Split_EMCV circRNA plasmids. Data for two biological replicates are shown.

### DETAILED DESCRIPTIONS OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. In the following passages, different aspects of the invention are defined in more detail. Each aspect so defined may be combined with any other aspect or aspects unless clearly indicated to the contrary. In particular, any feature indicated as being optional, preferred or advantageous may be combined with any other feature or features indicated as being optional, preferred or advantageous.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

In the following, some definitions of terms frequently used in this specification are provided. These terms will, in each instance of its use, in the remainder of the specification have the respectively defined meaning and preferred meanings.

As used in this specification and the appended claims, the singular forms “a”, “an”, and “the” include plural referents, unless the content clearly dictates otherwise.

The term “about” when used in connection with a numerical value is meant to encompass numerical values within a range having a lower limit that is 5% smaller than the indicated numerical value and having an upper limit that is 5% larger than the indicated numerical value.

The term “circular RNA” or “circRNA”, as used herein, refers to a type of RNA in which the ends of the RNA strand have been covalently joined to form a closed continuous loop. CircRNAs are generally formed by covalent binding of the 5′ site of an upstream exon with the 3′ site of the same or a downstream exon.

The terms “5′” and “3′”, as used herein, identify one end of a single-stranded nucleic acid molecule. The 5′ end is that end of the molecule which terminates in a 5′ phosphate group and the 5′ direction is the direction toward the 5′ end. Likewise, the 3′ end is that end of the molecule which terminates in a 3′ phosphate group and the 3′ direction is the direction toward the 3′ end. In the context of the present invention nucleic acid sequences are written with the 5′ end to the left and the 3′ end to the right (unless noted otherwise), in reference to the direction of DNA synthesis during replication (from 5′ to 3′), RNA synthesis during transcription (from 5′ to 3′), and the reading of mRNA sequence (from 5′ to 3′) during translation.

The term “open reading frame”, as used herein, refers to a portion of a nucleic acid sequence between the start and stop codons, which does not include a stop codon (which functions as a stop signal). A codon is a DNA or RNA sequence of three nucleotides that forms a unit of genomic information encoding a particular amino acid or signalling the termination of protein synthesis (stop codon). In the standard genetic code three different stop codons are known TAG, TAA or TGA on the DNA level or UAG, UAA and UGA on the RNA level. In variations of the standard genetic code other stop codons may exist. A start codon is the first codon of an RNA transcript (e.g. linear mRNA, circRNA) translated by a ribosome.

The term “backsplicing”, as used herein, refers to splicing at a reversed order (i.e. backsplicing) in which an upstream 3′ splicing site is joined with a downstream 5′ splicing site.

The term “backsplicing site”, as used herein, refers to a nucleotide sequence at the intron exon boundary (i.e. a splice site). The backsplicing site determines the position in which the pre-mRNA is spliced and allows the recruitment of the spliceosome, which is required for the backsplicing process. The sequence of the backsplicing site typically comprises two half-sites, whereas one half site is located on the intron and the other half-site on the exon. The spliced nucleotide sequence is split between the two half-sites of the backsplicing sites.

The term “Internal ribosomal entry site (IRES)”, as used herein, refers to regions in the RNAs that allow the internal initiation of translation in a cap-independent manner. An IRES element typically comprises a stretch of highly structured RNA containing several stem-loop structures. IRESs have initially been identified in picornavirus, but they are present in a variety of different virus. More recently IRES sequences have also been identified in many cellular mRNAs. Both types of IRES sequences (i.e. viral and cellular IRES sequences) generally can be used to practice the present invention.

Viral IRESs can be divided into four distinct classes based on two major criteria: first, the type of secondary and tertiary structures of their RNA elements and second, their mode of action for translation initiation (see Mailliot and Martin, R N A 2017, e1458).

Class 1 IRESs usually require all initiation factors except eIF4E and contain rather basic secondary structures consisting of short and long hairpins. Class 1 IRESs typically recruit the ribosome upstream of the coding region and rely on a classical 5′ to 3′ scanning to find the start codon. Class 1 IRESs are found for example in: Enterovirus A71 (EV-A71), Coxsackievirus B3 (CVB3), poliovirus (PV), and Human Rhino Virus 2 (HRV).

In contrast, class 2 IRESs promote direct tethering of the translation initiation machinery directly to the start codon and without any scanning step. Class 2 IRESs are found for example in: Picornaviridiae such as encephalomyocarditis virus (EMCV) and foot and mouth disease virus (FMDV).

Class 3 IRESs contain more sophisticated secondary and tertiary structures such as pseudoknots. They require only a small subset of translation initiation factors, namely eIF2, eIF3, and eIF5 to recruit the ribosome and load it directly on the AUG start codon without scanning. Class 3 IRESs are found for example in: Flaviviridae family such as hepatitis C virus (HCV) and classical swine fever virus (CSFV); Picornaviridae, such as porcine teschovirus and porcine enterovirus 8 (PEV8) or simian virus 2 (SV2).

Class 4 IRESs are the more compact and sophisticated IRESs in term of structural complexity; they usually contain several pseudoknots and do not require any translation initiation factors. They are the smallest IRESs known (usually less than 200 nucleotides). Class 4 IRESs are found for example in: Dicistroviridae such as cricket paralysis virus (CrPV), Israeli acute paralysis virus (IAPV), Platia Stali instestine virus (PSIV), or Taura syndrome virus (TSV).

The most preferred class of IRESs in the context of the present invention are class 1 and class 2 IRESs.

The term “inverted repeat (IR) element”, as used herein, refers to a nucleotide sequence that is followed downstream by its reverse complement sequence. The downstream nucleotide sequence may be the identical reverse complement sequence or may deviate therefrom.

The term “promoter”, as used herein, refers to a sequence of DNA to which proteins bind to initiate transcription of a single RNA transcript from the DNA downstream of the promoter. A promoter is typically located in the 5′ region of a gene, which does not code for the gene product, but controls its gene expression. In the promoter, transcription is initiated by the interaction of transcription factors and RNA polymerase. Generally, it is advantageous to select a promoter that is active in the desired type of host cell.

Promoters can be grouped into different categories based on the pattern of their activity. Some promoters are constitutive promoters that are active in essentially all tissues and do not require any particular stimuli to be active. Non limiting examples of constitutive promoters are: CMV, EF1a, EFS, CAG, CBh, CBA, MSCV, PGK, SFFV, SV40, and UBC. In a preferred embodiment the promoter is CMV. In another preferred embodiment the CMV promoter has a sequence according to SEQ ID NO: 005.

Other promoters are only active in certain tissues and are thus useful to restrict expression to specific tissues. Non limiting examples of tissue specific promoters that can be used in the present invention and the tissues they are preferably active in are provided in the following: Embryonic tissue: Nanog. Brain/Nervous-system/Spinal-cord: Nes, Tuba1a, Camk2a, SYN1, Hb9, Th, Thy1, NSE, GFAP(long), GFAP(short), Iba1, Prnp, Cnp, Retina: ProAl, hRHO, hBEST1, Grm6, Epidermis: K14, BK5, mTyr, Heart/embryonic heart: cTnT, aMHC(long), aMHC(short), Hcn4, Muscle: Myog, ACTA1, MHCK7, SM22a, EnSM22a, Desmin, Mb. Bone/Cartilage: Runx2, OC, Col1a1, Col2a1. Fat: aP2, Adipoq. Blood/Bone marrow: Liver: Afp, Alb, TBG. Mammary gland; MMTV, Wap. Pancreas: HIP, Pdx1, Ins2, Elastase-1, Kidney; NPHS2. Lung; SPB. Endothelial cells: CD144, Flt-1, ICAM-2, Endoglin, Hematopoietic cells: WASP, IFN-B, B19, CD14, CD43, CD45, CD68, Tiel, CD11b, OG-2. Tumors: TERT, E2F-1, OC, SLPI, Cox-2, CEA, AFP, LP-P. In a preferred embodiment the promoter is selected from SYN1, GFAP,

Other promoters require certain stimuli to be activated so that they initiate transcription. Non-limiting examples of inducible promoters are: TRE (tet-responsive promoter), CRE (cumate-responsive promoter)

In a preferred embodiment the promoter is selected from a constitutive, a tissue specific or an inducible promoter. In a preferred embodiment the promoter is a constitutive promoter. In a preferred embodiment the promoter is a tissue specific promoter. In a preferred embodiment the promoter is an inducible promoter.

The term “vector”, as used herein, refers to the vehicle by which a DNA or RNA sequence (e.g. a foreign gene) can be introduced into a host cell to transform the host and promote expression (e.g. transcription and translation) of the introduced sequence.

The terms “host cell”, as used herein, refers to a cell comprising the nucleic acid, the vector or the circRNA as described herein. The host cell can be an eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Preferably the host cell is a mammalian cell.

The term “branch point”, as used herein, refers to a nucleotide typically contained in an intronic heptamer sequence. The heptamer sequence, with the exception of the branch point, undergoes base-pairing with the spliceosome. The unpaired branchpoint is then required for the formation of a lariat intermediate during the splicing event. Typically, the branch point is an adenine.

The term “polypyrimidine tract”, as used herein, refers to a nucleotide sequence that is typically 15 to 20 base pairs in length and can promote the assembly of the spliceosome and thus assists in splicing.

### EMBODIMENTS

In the following different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

In a first aspect, the present invention provides a nucleic acid molecule encoding a circular RNA (circRNA), wherein the nucleic acid molecule comprises:
A) an expression cassette comprising:
   (i) a circRNA expression cassette comprising:
      (a) a nucleic acid comprising or consisting of a continuous or split open reading frame (ORF) encoding at least one protein, and
      (b) an internal ribosome entry site (IRES) operably linked to the ORF to direct translation of the ORF;
   (ii) a first backsplicing site that is positioned 5′ of the IRES; and
   (iii) a second backsplicing site that is positioned 3′ of the IRES;
and optionally
B) a first inverted repeat (IR) element positioned 5′ of the first backsplicing site and a second IR element positioned 3′ of the second backsplicing site, and/or a promoter operably linked to the expression cassette to direct expression of the expression cassette;
wherein the IRES is positioned within the circRNA expression cassette in that:
(I) the 5′ end of the IRES and the 3′ end of the first backsplicing site are separated by at least 50 nucleotides, preferably by at least 200 more preferably by at least 300 nucleotides, and
(II) the 3′ end of the IRES and the 5′ end of the second backsplicing site are separated by at least 300 nucleotides, preferably by at least 350 nucleotides.

In an alternative first aspect, the present invention provides a nucleic acid molecule encoding a circular RNA (circRNA), wherein the nucleic acid molecule comprises:
A) an expression cassette comprising:
   (i) a circRNA expression cassette comprising:
      (a) a nucleic acid comprising or consisting of a continuous or split open reading frame (ORF) encoding at least one protein, and
      (b) an internal ribosome entry site (IRES) operably linked to the ORF to direct translation of the ORF;
   (ii) a first backsplicing site that is positioned 5′ of the IRES; and
   (iii) a second backsplicing site that is positioned 3′ of the IRES;
and optionally
B) a first inverted repeat (IR) element positioned 5′ of the first backsplicing site and a second IR element positioned 3′ of the second backsplicing site, and/or a promoter operably linked to the expression cassette to direct expression of the expression cassette.

In the following the different elements of the nucleic acid of the different aspects of the invention are disclosed in more detail and particularly preferred embodiments are disclosed.

circRNA Expression Cassette and Expression Cassette

Without wishing to be bound by theory the inventors believe that an IRES site located in very close proximity to the backsplicing sites might interfere with backsplicing efficiency resulting in a reduced amount of circRNA being produced. This interference might be due to any secondary structures formed by the IRES that does not allow optimal access of the spliceosome to the linear pre-mRNA. Thus, arranging the IRES to be distanced from the backsplicing sites within the expression cassette is considered to be advantageous to improve backsplicing efficiency.

In a preferred embodiment, the IRES is positioned within the circRNA expression cassette in that the 5′ end of the IRES and the 3′ end of the first backsplicing site are separated by at least 50 nucleotides, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 350, at least 400, at least 450, at least 475, at least 500 nucleotides. In a preferred embodiment the 5′ end of the IRES and the 3′ end of the first backsplicing site are separated by at least 160 nucleotides. In a preferred embodiment the 5′ end of the IRES and the 3′ end of the first backsplicing site are separated by at least 475 nucleotides.

In a preferred embodiment, the IRES is positioned within the circRNA expression cassette in that the 3′ end of the IRES and the 5′ end of the second backsplicing site are separated by at least 300, at least 310, at least 320, at least 330, at least 340, at least 350 nucleotides.

In a preferred embodiment, the IRES is positioned within the circRNA expression cassette in that the 3′ end of the IRES and the 5′ end of the second backsplicing site are separated by at least 50 nucleotides, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290 nucleotides. In a preferred embodiment, the IRES is positioned within the circRNA expression cassette in that the 3′ end of the IRES and the 5′ end of the second backsplicing site are separated by at least 50 nucleotides.

In a preferred embodiment, the IRES is positioned within the circRNA expression cassette in that the 5′ end of the IRES and the 3′ end of the first backsplicing site are separated by at least 50 nucleotides, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 350 nucleotides (preferably at least 250 nucleotides) and the 3′ end of the IRES and the 5′ end of the second backsplicing site are separated by at least 300, at least 310, at least 320, at least 330, at least 340, at least 350 nucleotides (preferably at least 300).

In a preferred embodiment, the IRES is positioned within the circRNA expression cassette in that the 5′ end of the IRES and the 3′ end of the first backsplicing site are separated by at least 50 nucleotides and the 3′ end of the IRES and the 5′ end of the second backsplicing site are separated by at least 350 nucleotides.

In a preferred embodiment, the IRES is positioned within the circRNA expression cassette in that the 5′ end of the IRES and the 3′ end of the first backsplicing site are separated by at least 50 nucleotides and the 3′ end of the IRES and the 5′ end of the second backsplicing site are separated by at least 680 nucleotides.

In a preferred embodiment, the IRES is positioned within the circRNA expression cassette in that the 5′ end of the IRES and the 3′ end of the first backsplicing site are separated by at least 250 nucleotides and the 3′ end of the IRES and the 5′ end of the second backsplicing site are separated by at least 480 nucleotides.

In a preferred embodiment, the IRES is positioned within the circRNA expression cassette in that the 5′ end of the IRES and the 3′ end of the first backsplicing site are separated by at least 360 nucleotides and the 3′ end of the IRES and the 5′ end of the second backsplicing site are separated by at least 380 nucleotides.

In a preferred embodiment, the IRES is positioned within the circRNA expression cassette in that the 5′ end of the IRES and the 3′ end of the first backsplicing site are separated by at least 650 nucleotides and the 3′ end of the IRES and the 5′ end of the second backsplicing site are separated by at least 90 nucleotides.

In a preferred embodiment, the IRES is positioned within the circRNA expression cassette in that the 5′ end of the IRES and the 3′ end of the first backsplicing site are separated by at least 300 nucleotides and the 3′ end of the IRES and the 5′ end of the second backsplicing site are separated by at least 300 nucleotides.

In a preferred embodiment, the IRES is positioned within the circRNA expression cassette in that the 5′ end of the IRES and the 3′ end of the first backsplicing site as well as the 3′ end of the IRES and the 5′ end of the second backsplicing site have both a distance of at least 50, at least 100, at least 150, at least 200, at least 250, at least 300, at least 350, at least 400 nucleotides of each other.

In a preferred embodiment, the IRES is positioned within the circRNA expression cassette in that the 5′ end of the IRES and the 3′ end of the first backsplicing site have a distance of 50 to 650 nucleotides (preferably 200 to 400 nt, more preferably 250 to 365 nt) of each other.

In a preferred embodiment, the IRES is positioned within the circRNA expression cassette in that the 5′ end of the IRES and the 3′ end of the first backsplicing site have a distance of 59 to 650 nucleotides (preferably 255 to 363 nt) of each other.

In a preferred embodiment, the IRES is positioned within the circRNA expression cassette in that the 3′ end of the IRES and the 5′ end of the second backsplicing site have a distance of 300 to 700 nucleotides (preferably 350 to 600 nt, more preferably 350 to 500 nt) of each other.

In a preferred embodiment, the IRES is positioned within the circRNA expression cassette in that the 3′ end of the IRES and the 5′ end of the second backsplicing site have a distance of 381 to 685 nucleotides (preferably 381 to 489 nt) of each other.

In a preferred embodiment, the IRES is positioned within the circRNA expression cassette in that the 5′ end of the IRES and the 3′ end of the first backsplicing site have a distance of 50 to 650 nucleotides (preferably 200 to 400 nt, more preferably 250 to 365 nt) of each other and the 5′ end of the IRES and the 3′ end of the first backsplicing site have a distance of of 300 to 700 nucleotides (preferably 350 to 600 nt, more preferably 350 to 500 nt) of each other.

In a preferred embodiment, the IRES is positioned within the circRNA expression cassette in that the 5′ end of the IRES and the 3′ end of the first backsplicing site have a distance of 59 to 650 nucleotides (preferably 255 to 363 nt) of each other and the 3′ end of the IRES and the 5′ end of the second backsplicing site have a distance of 381 to 685 nucleotides (preferably 381 to 489 nt) of each other.

In a preferred embodiment, the IRES is positioned within the circRNA expression cassette in that the IRES is located in the middle of the circRNA expression cassette. In one embodiment, the nucleotides having equal distance to the first and second backsplicing site is part of the IRES sequence. In one embodiment the sequences flanking the IRES sequence in the circRNA expression cassette differ by no more than 50 nucleotides.

Yet another possibility to distance the IRES sequence from the backsplicing sites is the use of a nucleotide sequence that is not part of the ORF, i.e. the use of a spacer sequence. In a preferred embodiment the circRNA expression cassette includes a spacer sequence thus separating the IRES and the first backsplicing site. In a preferred embodiment the spacer sequence is positioned 5′ of the IRES sequence and 3′ of the first backsplicing site. In another preferred embodiment the spacer sequence is positioned 3′ of the IRES sequence and 5′ of the second backsplicing site. In yet another preferred embodiment the circRNA expression cassette comprises two spacer sequences. Preferably the first spacer sequence is positioned 5′ of the IRES sequence and 3′ of the first backsplicing site and the second spacer sequence is positioned 3′ of the IRES sequence and 5′ of the second backsplicing site. In a preferred embodiment the spacer sequence has a length of 50 to 500 nucleotides, preferably 100 to 400 nucleotides, more preferably 150 to 300 nucleotides, most preferably 200 to 250 nucleotides. In a preferred embodiment the spacer sequence has a length of 50, 100, 200 or 400 nucleotides.

In yet another embodiment the spacer sequence may encode a protein.

In a preferred embodiment, the IRES is CVB3 and the IRES is positioned within the circRNA expression cassette in that:
(I) the 5′ end of the IRES and the 3′ end of the first backsplicing site are separated by at least 50 nucleotides, preferably by at least 200 more preferably by at least 300 nucleotides, and
(II) the 3′ end of the IRES and the 5′ end of the second backsplicing site are separated by at least 50 nucleotides, preferably by at least 100, at least 150, at least 200, at least 250 nucleotides.

### Structure of the Open Reading Frame (ORF)

The ORF comprised in the circRNA expression cassette may be arranged as a continuous ORF located 3′ or 5′ of the IRES or as a split ORF wherein the ORF is split (preferably at a sequence that creates a half site of a backsplicing site at the position of the split) so that the two parts of the split ORF are flanking the IRES in the circRNA expression cassette.

A split-ORF has several advantageous properties in the context of the present invention. First, the split ORF design distances the IRES from the backsplicing sites, which is considered to improve the backsplicing efficiency and thereby increases the circRNA yield, without the need for further sequence elements, i.e. spacer sequences. Another advantageous property is that the protein encoded in the ORF can only be translated from a resulting circRNA and not from a linear RNA that may arise from the nucleic acid molecule if circularization fails or is incomplete. Thus, it is safeguarded that only proteins encoded in circRNA are translated.

In a preferred embodiment of the first aspect of the present invention, the split ORF comprises two parts and the first part of the split ORF comprises a STOP codon and is positioned 5′ of the IRES and the second part of the split ORF comprises a START codon and is positioned 3′ of the IRES. In a circRNA produced from the nucleic acid of the first aspect of the invention the split ORF will be reassembled by the circularization of the circRNA to result in a continuous ORF in the closed loop of the circRNA.

In a preferred embodiment wherein the circRNA expression cassette comprises a split-ORF the second half site of the first backsplicing site and the first half site of the second backsplicing site is located within the two parts of the split ORF. In other words, they are part of the protein coding sequence in the ORF. This is advantageous because otherwise any residual sequence left by the splicing event would be within the translated sequence of the ORF and might interfere with protein translation and/or folding due to additional nucleotides within the sequence.

In another preferred embodiment the ORF is a continuous ORF. Preferably a circRNA expression cassette comprising a continuous ORF further includes a non-coding spacer sequence that distances the IRES from the backsplicing site. Exemplary usable spacer sequences are described above.

In a preferred embodiment the circRNA expression cassette comprises a continuous ORF and further comprises one or more non-coding nucleotide sequence(s) positioned at the 5′ end and/or 3′ end of the continuous ORF.

In a preferred embodiment, the circRNA expression cassette comprises a split ORF and further comprises one or more non-coding nucleotide sequence(s) positioned at the 5′ end and/or the 3′ end of the IRES.

In a preferred embodiment, the circRNA expression cassette further comprises one or more (e.g. 1, 2, 3, 4) additional ORFs.

In a preferred embodiment, the circRNA expression cassette further comprises one or more (e.g. 1, 2, 3, 4) additional IRES.

In a preferred embodiment, the circRNA expression cassette further comprises an additional coding nucleotide sequence attached to one or more ORF, preferably encoding a sequence that can be used to detect or capture the protein encoded in the one or more ORF(s).

In a preferred embodiment the ORF encodes at least one therapeutic protein, therapeutic peptide, antigenic protein, or antigenic peptide. In general, any type of peptide or protein may be encoded by the ORF.

### Backsplicing Sites

The nucleic acid of the invention comprises at least two backsplicing sites that are located upstream (i.e. 5′) and downstream (i.e. 3′) of the IRES. The backsplicing sites are important to allow formation (i.e. circularization) of the circRNA encoded by the nucleic acid of the first aspect of the invention. The backsplicing sites determine the position in which the pre-mRNA is spliced and allows the recruitment of the spliceosome, which is required for the backsplicing process. Typically, a backsplicing site comprises two half-sites, wherein the nucleic acid is split between the two half-sites during splicing. In this context the term ‘splice acceptor (SA)’ refers to the 3′ end of an intron. The term ‘splice donor (SD)’ refers to the 5′ end of an intron. In a preferred embodiment the first backsplicing site comprises the splice acceptor and the second backsplicing site comprises the splice donor.

In a preferred embodiment the backsplicing sites comprise a nucleotide sequence that allows for spliceosome assisted backsplicing of the encoded circRNA. In a preferred embodiment the backsplicing sites require a spliceosome to yield a circRNA encoded by the circRNA expression cassette.

In a preferred embodiment the first backsplicing site is comprising two half-sites. In one embodiment the first and the second half-sites of the first backsplicing site are positioned 5′ of the circRNA expression cassette. In another embodiment, the first half-site of the first backsplicing site is positioned 5′ of the circRNA expression cassette and the second half-site of the first backsplicing site is part of the circRNA expression cassette. This embodiment is preferred in case of the ORF being a split ORF. Preferably the second half-site of the first backsplicing site is part of the ORF sequence.

In a preferred embodiment, the circRNA resulting from backsplicing the nucleic acid molecule of the first aspect of the invention does not comprise any sequence derived from the backsplicing site that is not part of the ORF or the IRES sequence.

In a preferred embodiment, the second backsplicing site is comprising two half-sites. In one embodiment the first and the second half-sites of the second backsplicing site are positioned 3′ of the circRNA expression cassette. In another embodiment, the first half-site of the second backsplicing site is part of the circRNA expression cassette. This embodiment is preferred in case of the ORF being a split ORF. Preferably, the first half-site of the second backsplicing site is part of the ORF in the case of a split ORF, and the second half-site of the second backsplicing site is positioned 3′ of the circRNA expression cassette.

In a preferred embodiment, the first and second backsplicing sites are comprising two half-sites, respectively. In one embodiment the first and the second half-sites of the first backsplicing site are positioned 5′ of the circRNA expression cassette and the first and the second half-sites of the second backsplicing site are positioned 3′ of the circRNA expression cassette. In another embodiment, the first half-site of the first backsplicing site is positioned 5′ of the circRNA expression cassette and the second half-site of the first backsplicing site is part of the circRNA expression cassette and the first half-site of the second backsplicing site is part of the circRNA expression cassette and the second half-site of the second backsplicing site is positioned 3′ of the circRNA expression cassette.

In a preferred embodiment, the first half site of the first backsplicing site comprises or consists of the nucleotides AG. In another preferred embodiment the first half site of the first backsplicing site comprises or consists of the nucleotide sequence CAG. In another preferred embodiment the first half site of the first backsplicing site is the splice acceptor. In another preferred embodiment, the second half site of the first backsplicing site begins with the nucleotide G.

In a preferred embodiment, the second half site of the second backsplicing site comprises or consists of the nucleotides GT. In another preferred embodiment the second half site of the second backsplicing site comprises or consists of the nucleotide sequence GTAAGT.

In another preferred embodiment the second half site of the second backsplicing site is the splice donor. In another preferred embodiment, the first half site of the second backsplicing site comprises or consists of the nucleotide sequence CAG or AAG.

In a preferred embodiment, the first half site of the first backsplicing site comprises or consists of the nucleotides AG; and the second half site of the second backsplicing site comprises or consists of the nucleotides GT. In a preferred embodiment the first half site of the first backsplicing site comprises or consists of the nucleotides CAG; and the second half site of the second backsplicing site comprises or consists of the nucleotides GTAAGT.

In a preferred embodiment, the backsplicing sites are consensus sequences for U2 (major class) introns in pre-mRNA generally conform to the following consensus sequences: 3′ splice sites: CAG|G and 5′ splice sites: MAG|GTRAGT where M is A or C, R is A or G and indicates the intron exon boundary. In another preferred embodiment the splice sites have a sequence as indicated by the similarity matrices for human U2 introns as for example disclosed in Zhang Hum Mol Genet. 1998 May; 7(5):919-32, which is incorporated herein by reference.

In a preferred embodiment the sequence of the ORF is modified to create a backsplicing site. Preferably the modification introduces only silent mutations (i.e. without changing the amino acid sequence of the encoded protein).

In a preferred embodiment the first backsplicing site has the nucleotide sequence CAGGT. In another preferred embodiment the first backsplicing site has a sequence being at least 85%, at least 90%, at least 95% or at least 99% identical to the nucleotide sequence CAGGT.

In a preferred embodiment the second backsplicing site has the nucleotide sequence AGGTA. In another preferred embodiment the second backsplicing site has a sequence being at least 85%, at least 90%, at least 95% or at least 99% identical to the nucleotide sequence AGGTA.

### Internal Ribosome Entry Site (IRES)

circRNAs are covalently linked forming a closed loop and thus lack a 5′ terminus and must rely on cap-independent mechanisms to initiate translation of encoded proteins. Internal ribosome entry sites (IRES) are RNA sequences that recruit the 40S ribosomal subunit through cap-independent mechanisms and thus allow to initiate translation. These elements typically adopt complex secondary RNA structures, which serve as the anchoring site for the ribosome.

The nucleic acid molecule of the invention therefore comprises an IRES in the circRNA expression cassette. The IRES is operably linked to the ORF so that the at least one protein encoded in the ORF can be translated from the circRNA. Without wishing to be bound by theory the inventors believe that the complex secondary structure of an IRES or IRES-associated proteins may interfere with the spliceosome if the IRES is in close proximity to the backsplicing site. Although IRES are diverse with regard to their nucleic acid sequence they have in common that they typically have a complex secondary structure. Therefore, interference of an IRES with the spliceosome is independent of a particular IRES, but rather depends on the secondary structure common for IRES in general.

In a preferred embodiment the IRES is a viral IRES or a cellular IRES, preferably a viral IRES. In a preferred embodiment, the IRES is a class 1 or class 2 IRES as defined above. Non limiting examples of class 1 IRES are found in Enterovirus A71 (EV-A71), Coxsackievirus B3 (CVB3), poliovirus (PV), and Human Rhino Virus 2 (HRV), preferably CVB3. Non-limiting examples of class 2 IRES are found in Picornaviridiae such as encephalomyocarditis virus (EMCV) and foot and mouth disease virus (FMDV), preferably EMCV.

In a preferred embodiment the IRES is a class 1 IRES. In another preferred embodiment the IRES is a class 2 IRES. In another preferred embodiment the IRES is a class 3 IRES. In another preferred embodiment the IRES is a class 4 IRES.

In a preferred embodiment the IRES is an IRES sequence from Coxsackievirus (CVB), more preferably CVB3. In a more preferred embodiment, the IRES has the sequence according to SEQ ID NO: 001. In another preferred embodiment the IRES has a sequence being at least 50%, at least 75%, at least 85%, at least 90%, at least 95% or at least 99% identical to the sequence of SEQ ID NO: 001. In a preferred embodiment the IRES has a sequence being at least 75% identical to the sequence of SEQ ID NO: 001. In a preferred embodiment the IRES has a sequence according to the sequence of SEQ ID NO: 001.

In a preferred embodiment the IRES is an IRES sequence from encephalomyocarditis virus (EMCV). In a more preferred embodiment, the IRES has the sequence according to SEQ ID NO: 002. In another preferred embodiment the IRES has a sequence being at least 85%, at least 90%, at least 95% or at least 99% identical to the sequence of SEQ ID NO: 002.

In one embodiment the IRES is a viral IRES, preferably of a virus selected from the viral families of Adenoviridae, Arenaviridae, Birnaviridae, Chrysoviridae, Coronaviridae, Dicistroviridae, Filoviridae, Flaviviridae, Hepadnaviridae, Herpesviridae, Hypoviridae, Iflaviridae, Luteoviridae, Orthomyxoviridae, Papillomaviridae, Paramyxoviridae, Parvoviridae, Picornaviridae, Pneumoviridae, Polyomaviridae, Potyviridae, Reoviridae, Retroviridae, Rhabdoviridae, Secoviridae, Tombusviridae, Totiviridae, Virgaviridae.

In another preferred embodiment the IRES is a variant of a viral IRES (preferably CVB3 or EMCV, more preferably CVB3), which has a nucleotide sequence that is at least 80%, at least 85%, at least 90%, at least 95%, at least 99% identical to the nucleotide sequence of the viral IRES.

### Inverted Repeat (IR) Elements

Although not necessarily required for backsplicing of circRNA the presence of inverted repeat elements flanking the splice sites are known to improve circRNA yield. It is believed that the inverted repeat elements bring the splice sites into close proximity to facilitate backsplicing of the circRNA.

In the context of the present invention IR elements positioned 5′ of the first backsplicing site (i.e. first IR element) and 3′ of the second backsplicing site (i.e. second IR element) are used to further improve backsplicing efficiency.

Generally, any nucleotide sequence that is followed downstream by its reverse complement sequence may be used as IR element in the present invention. In some embodiment the downstream sequence (i.e. second IR element) is not identical to the reverse complement of the upstream sequence (i.e. first IR element).

In some embodiments the downstream nucleotide sequence (i.e. second IR element) is at least 60%, at least 65%, at least 70%, at least 75%, at least 80%, at least 85%, at least 90%, at least 95% or at least 99% identical to the reverse complement of the upstream sequence (i.e. first IR element). In a preferred embodiment the first and the second IR element comprise a sequence being at least 80%, preferably at least 90%, more preferably at least 95% identical to the reverse complementary sequence of each other.

In a preferred embodiment the downstream nucleotide sequence differs in 5, 4, 3, 2 or 1 nucleotide(s) from the reverse complement of the upstream sequence.

In a preferred embodiment the IR element has a length of 50 to 1000, preferably 200 to 600, more preferably 300 to 500 nucleotides. In another preferred embodiment the IR element has a length of 200 to 400 nucleotides.

IR elements used in the present invention may be IR elements known in the art or those derived from genes such as AC004076.9, ACYP2, AMD1, ARHGAP10, ARHGAP12, ARHGEF12, ARHGEF28, ASAP1, ASH1L, ASXL1, ATXN2, BMPR2, BRWD1, BTBD10, CBFA2T2, CCDC126, CCDC134, CCDC66, CCDC7, CCDC9, CCNB1, CDK13, CFLAR, CHD9, CLIP2, CLNS1A, CNN2, COA1, CORO1C, CREBBP, CRKL, CTB-43P18.3,SNHG4, DCUN1D4, DEK, DHRS3, DLG1, DOPEY2, DYNC1H1, ELF2, EMC2, EPHB4, EPS15, ERC1, ETFA, EXOSC1, FAM13B, FARSA, FBXO7, FGD4, FGD6, FKBP3, FKBP8, FNTA, FOXK2, GAPVD1, GBAS, GDI2, GLIS2, GLS, GON4L, GRHPR, HERC1, HIPK3, HNRNPM, HOOK3, HP1BP3, HPS5, HTT, HUWE1, IARS, ILKAP, IQGAP1, KDM1A, KIAA0368, KIAA1429, KIAA1841, KLHL8, KMT2C, Laccase, LMBR1, LRCH3, LZIC, MAP3K1, MARK4, MBOAT2, MCU, MED13L, METTL3, MGA, MGEA5, MITD1, MORC3, MRPS35, MYO9B, NCOA2, NFAT5, NFATC3, NFX1, NUDC, NUP54, PAFAH1B2, PAIP2, PCMT1, PDCD11, PDE8A, PDS5A, PHC3, PHLDB2, PLEKHM1, PLEKHM3, PLOD2, PMS1, PNN, POLR2A, POMT1, PPP6R2, PROSC, PRRC2B, PSEN1, PSMA7, PTP4A2, PTPN12, QKI, R3HDM1, RAB6A, RALBP1, RARS, RBM23, RBM33, RBM39, RELL1, REPS1, RERE, RHOBTB3, RLF, RNF19B, SDHAF2, ZRANB1, FAM228B, RPRD1B, RPS6KC1, RSF1, RSRC1, RTN4, RYK, SAFB2, SAMD4A, SCAF8, SCARFI, SCYL2, SDF4, SEC31A, SENP6, SIPA1L1, SKA3, SLC38A1, SLTM, SMARCA5, SMC3, SMO, SNX25, SOBP, SOS2, SPIDR, SPPL3, SRSF4, STAM, STK3, STX6, TERF2, TIMMDC1, TMED2, TMEM138, TMEM165, TMEM181, TNPO1, TNPO3, TOP1, TTC39C, TTLL1, UBA2, UBAP2, UBE2K, UBQLN1, UBR5, UBXN2A, UBXN7, UHRF2, UIMC1, URI1, UTP18, UTRN, VAMP3, VAPB, VMP1, WDR78, XPO1, YTHDF2, YWHAE, YYlAP1, ZBTB46, ZCCHC11, ZCCHC6, ZFAND6, ZFX, ZKSCAN1, ZMYM4, ZNF124, ZNF236, ZNF394, ZNF430, ZNF652, ZNF720, ZNF91. Preferably the IR elements are selected from HIPK3 and ZKSCAN1.

In a preferred embodiment the IR elements are derived from genes with IR element derived circRNAs.

In a preferred embodiment the IR elements are short interspersed nuclear elements (SINE) elements.

In a preferred embodiment the IR elements are Alu-repeat elements.

In a preferred embodiment the first IR element has a sequence according to SEQ ID NO: 003. In another preferred embodiment the first IR element has a sequence being at least 85%, at least 90%, at least 95% or at least 99% identical to the sequence of SEQ ID NO: 003. In a preferred embodiment the second IR element has a sequence according to SEQ ID NO: 004. In another preferred embodiment the second IR element has a sequence being at least 85%, at least 90%, at least 95% or at least 99% identical to the sequence of SEQ ID NO: 004.

In a preferred embodiment the first IR element has a sequence as comprised in the disclosed constructs of the invention. In another preferred embodiment the first IR element has a sequence being at least 85%, at least 90%, at least 95% or at least 99% identical to the sequence as comprised in the disclosed constructs of the invention.

In a preferred embodiment the second IR element has a sequence as comprised in the disclosed constructs of the invention. In another preferred embodiment the second IR element has a sequence being at least 85%, at least 90%, at least 95% or at least 99% identical to the sequence as comprised in the disclosed constructs of the invention.

### CircRNA

The nucleic acid of the invention may in general encode any type of circRNA. In a preferred embodiment the circRNA encoded by the nucleic acid of the invention or the circRNA of the second aspect of the invention has a length of 50 nt to 5000 nt, preferable has a length of 200 nt to 2000 nt.

In a preferred embodiment the circRNA encoded by the nucleic acid of the invention or the circRNA of the second aspect of the invention does not contain any sequences of the backsplicing sites that do not form part of the ORF or IRES sequence.

In a preferred embodiment, the circRNA encoded by the nucleic acid of the invention or the circRNA of the second aspect of the invention does not encode a fluorescent protein, preferably does not encode a green or red fluorescent protein.

In a preferred embodiment, the circRNA encoded by the nucleic acid of the invention or the circRNA of the second aspect of the invention does encode a therapeutic protein, a therapeutic peptide, an antigenic protein or an antigenic peptide.

In a preferred embodiment, the circRNA has a GC content of between 30%-70%, preferably 40% to 60%, more preferably 50% to 60%.

In a preferred embodiment, the circRNA does not contain IR elements.

In a preferred embodiment, the circRNA is devoid of perfect miRNA target sites. This embodiment is particularly useful to prevent miRNA-mediated endo-cleavage.

In a preferred embodiment, the circRNA is devoid of cryptic splice sites.

### Further Elements of the Nucleic Acid of the Invention

The nucleic acid of the invention may further comprise elements that have a function in regulation of expression, stability etc.

In a preferred embodiment the nucleic acid comprises a promoter operably linked to the expression cassette to direct expression of the expression cassette. In another embodiment the nucleic acid further comprises an enhancer. Examples of promoters and enhancers used in the nucleic acid of the invention include early promoter and enhancer of SV40 (Mizukami T. et al. 1987), LTR promoter and enhancer of Moloney mouse leukemia virus (Kuwana Y et al. 1987), immediate early promoter and enhancer of cytomegalovirus (CMV) and the like.

In another preferred embodiment, the nucleic acid molecule further comprises a branch point.

In another preferred embodiment, the nucleic acid molecule further comprises a polypyrimidine tract.

### Vector of the Invention

A third aspect of the invention refers to a vector comprising the nucleic acid molecule according to the first aspect of the invention or the circRNA according to the second aspect of the invention.

A vector in the sense of the present invention refers to any type of element that allows to comprise the nucleic acid or the circRNA of the invention. Any suitable vector, such as a plasmid, cosmid, episome, liposome, exosome, artificial chromosome, phage or a viral vector, can be used in the invention.

The terms “vector”, “cloning vector” and “expression vector” mean the vehicle by which a DNA or RNA sequence (e.g. the nucleic acid or circRNA of the invention) can be introduced into a host cell, so as to transform the host and promote expression (e.g. transcription, circularization and translation) of the introduced nucleic acid or circRNA.

Such vectors may comprise regulatory elements, such as a promoter, enhancer, terminator and the like, to cause or direct expression of said nucleic acid upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of cytomegalovirus (CMV) and the like.

Any expression vector for animal cell can be used, so long as the nucleic acid of the invention can be inserted and expressed. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other examples of plasmids include replicating plasmids comprising an origin of replication, and the like.

### Host Cell of the Invention

A fourth aspect of the present invention relates to a host cell that comprises the nucleic acid, the circRNA and/or the vector according to the invention (preferably by transformation, transduction or transfection of the host cell).

The term “transformation” originally refers to a naturally occurring process of gene transfer into a host cell which involves absorption of the genetic material, such as nucleic acids, for instance, DNA or RNA, by a cell through cell membrane, so that the host cell will express the introduced gene or sequence to produce a desired substance. There are two types called as natural transformation and artificial or induced transformation. The artificial or induced method of transformation is done under laboratory condition.

A host cell that receives and expresses foreign nucleic acids, such as DNA or RNA, by the process of a transformation has been “transformed”.

The term “transfection” as used herein refers to a mode of gene transfer involving creation of pores on the cell membrane of the host cell enabling the host cell to receive the foreign genetic material. Typically, transfection refers to a transformation of eukaryotic cells, such as insect or mammalian cells. Chemical mediated transfection involves use of, for instance, calcium phosphate or cationic polymers or liposomes. Non-chemical mediated transfection methods are typically electroporation, sonoporation, impalefection, optical transfection or hydro dynamic delivery. Particle based transfection uses gene gun techniques where a nanoparticle is used to transfer the nucleic acid to host cell or by another method called as magnetofection. Nucleofection and use of heat shock are the other evolved methods for successful transfection. A host cell that receives foreign nucleic acids via a transfection method has been “transfected”.

The term “transduction” as used herein is generally understood to relate to the transfer of foreign nucleic acids, such as DNA or RNA, into a cell by a virus or viral vector. A host cell that receives and expresses foreign nucleic acids, such as DNA or RNA, by a virus or viral vector has been “transduced”.

### Pharmaceutical Composition

A fifth aspect of the invention refers to a pharmaceutical composition comprising the nucleic acid, the circRNA, the vector and/or the host cell of the invention and a pharmaceutically acceptable carrier.

The terms “pharmaceutical composition” or “therapeutic composition” as used herein refer to a compound or composition capable of inducing a desired therapeutic effect when properly administered to a subject.

In some embodiments, the subject may also be referred to as patient.

Such therapeutic or pharmaceutical compositions may comprise a therapeutically effective amount of a nucleic acid, a circRNA, a vector and/or a host cell of the invention or further comprising a therapeutic agent, in admixture with a pharmaceutically or physiologically acceptable formulation agent selected for suitability with the mode of administration.

The nucleic acid, circRNA, vector and/or host cell of the invention will usually be supplied as part of a sterile, pharmaceutical composition which will normally include a pharmaceutically acceptable carrier.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

A “pharmaceutically-acceptable carrier” may also be referred to as “pharmaceutically acceptable diluent” or “pharmaceutically acceptable vehicles” and may include solvents, bulking agents, stabilizing agents, dispersion media, coatings, antibacterial and antifungal agents, isotonic and absorption delaying agents, and the like which are physiologically compatible. Accordingly, in one embodiment the carrier is an aqueous carrier.

The form of the pharmaceutical compositions, the route of administration, the dosage and the regimen naturally depend upon the condition to be treated, the severity of the illness, the age, weight, and gender of the patient, the desired duration of the treatment etc. This pharmaceutical composition may be in any suitable form, (depending upon the desired method of administering it to a patient). It may be provided in unit dosage form, will generally be provided in a sealed container and may be provided as part of a kit. Such a kit would normally (although not necessarily) include instructions for use. It may include a plurality of said unit dosage forms.

Empirical considerations, such as the biological half-life or the bioavailability, generally will contribute to the determination of the dosage. Frequency of administration may be determined and adjusted over the course of therapy. Alternatively, sustained continuous release formulations may be appropriate.

### Other Aspects of the Invention

The present invention further includes a method for producing circular mRNA (circRNA), said method comprising the steps of introducing the nucleic acid molecule of the first aspect of the invention or the vector of the third aspect of the invention in a eukaryotic cell and optionally purifying circRNA from the cell.

In another aspect of the invention the method for producing circular mRNA (circRNA) comprises the steps of contacting the nucleic acid molecule of the first aspect of the invention or the vector according to the third aspect of the invention with isolated RNA polymerase II.

In yet another aspect the present invention refers to a method for producing a recombinant protein. The method comprises introducing the nucleic acid molecule of the first aspect of the invention, the circRNA according to the second aspect or the vector according to the third aspect into a eukaryotic cell and optionally purifying the recombinant protein encoded by the ORF.

In yet another aspect of the invention a circRNA obtained by the method of the invention is claimed.

### EXAMPLES

The present invention provides a nucleic acid encoding circRNA that utilizes spliceosome-based backsplicing for cellular circRNA expression. The flanking regions of the highly expressed circRNA, derived from the HIPK3 locus, were used to facilitate backsplicing. Additionally, introduction of an IRES element is required to facilitate circRNA cap-independent protein translation. The CVB3 IRES was found to be superior for protein expression compared to the EMCV IRES, although both IRES elements result in improved circRNA expression using the nucleic acid design of the present invention. Surprisingly, the inventors observed that positioning of the IRES within the circRNA expression cassette greatly impacts circRNA expression on both an RNA and protein level, suggesting improved backsplicing. The positioning of the IRES element in proximity to the flanking regions of the circRNA cassette was found to suppress circRNA expression. Without wishing to be bound by theory the inventors thus hypothesize that the highly structured IRES element may interfere with splice site recognition and assembly of the spliceosome thus inhibiting the backsplicing reaction and circRNA production. This is supported by the observation that increasing the distance of the IRES from the splice sites through the addition of non-coding spacer sequences between the SA and IRES restores circRNA production based in a distance-dependent manner. Increasing the distance between both features correlates positively with circRNA expression.

For transgene protein expression, the incorporation of additional non-coding sequences appears superfluous as they provide no additional function other than facilitating efficient backsplicing and may reduce the payload/insert capacity for expression of a gene of interest. To overcome this a split ORF circRNA design was examined. The transgene payload is split at either a CAGG or AAGG motif within the ORF to best mirror the endogenous splicing reaction. The inventors observed that splitting the ORF greatly improved circRNA production compared to insertion of the continuous ORF immediately upstream or downstream of the IRES. Additionally, the inventors found that splitting the ORF more centrally and thus increasing the distance between the IRES and the splice sites improves circRNA production, when compared to a continuous ORF design and when compared to splitting the ORF in the 5′ and 3′ends. Thus, supporting the hypothesis that proximity of the IRES to the splice sites negatively impacts circRNA backsplicing. In further support of this, splitting the IRES element itself and inserting the ORF within the IRES was also found to inhibit efficient circRNA production.

Finally, the split ORF design was found to be superior across multiple transgenes when compared to the continuous ORF design. This suggests that the circRNA expression design of the present invention is a powerful tool for expression of transgenes.

### General Methods Applied in the Examples

### Constructs, Cell Culture & Treatments:

Plasmids were synthesized by Genscript using the pcDNA3.1 backbone vector for mammalian expression. The A375 human melanoma cell line was obtained from (ATCC) and maintained in Dulbecco's modified Eagle's medium (Thermo Fisher Scientific) supplemented with 10% fetal bovine serum (Thermo Fisher Scientific) and antibiotics (100 U/ml penicillin and 100 mg/ml streptomycin; Thermo Fisher Scientific, Inc.). All cell types were incubated in a 5% CO2 humidified incubator at 37° C.

Cells were seeded ~150,000 cells per well of a 6-well dish 24 hrs prior to transfection. Cells were transfected using Lipofectamine 3000 following manufacturers guidelines. Briefly 2 ug of circRNA or pcDNA3.1 linear expression vectors were transfected per well. Cells were incubated with transfection mix overnight before media change. RNA and protein were harvested 48 hrs post transfection unless otherwise specified. For all experiments, cells were harvested by washing in 1×PBS and subsequent centrifugation at 1200 rpm at 4° C. for 4 min. 66.6% of the harvested cells was used for RNA isolation, which was carried out using TRIzol Reagent (Thermo Fisher Scientific) according to manufacturer's protocol.

### RT-PCR and RT-qPCR:

One g of DNase-treated total RNA was reverse transcribed using the M-HLV Reverse Transcriptase kit (Thermo Fisher Scientific) according to manufacturer's protocol with the use of random hexamers to prime the reaction. In case of RT-PCR, the reaction was conducted with 25 cycles of PCR with or without RT enzyme. The products were visualized by 1% agarose gel electrophoresis and verified using Sanger sequencing. For quantitative PCR, cDNA was mixed with Platinum SYBR Green I Master kit (Invitrogen) and ran on 7500 Fast Real-Time PCR System (Applied Biosystem). The reactions were carried out in technical triplicates. The obtained Ct values for each triplicate were transformed (2-Ct) and averaged (a). All samples were normalized to GAPDH. The results were visualized as bar plots using GraphPad (Prism 7) where individual biological replicates are shown, and the standard deviation is plotted as a bar.

### Western Blotting:

Cells were harvested in 1×PBS and centrifuged at 1200 rpm at 4° C. for 5 min. For cell lysis, the cell pellet was collected and resuspended in 50 μL RIPA buffer supplemented with protease inhibitors. Lysate was incubated for 20 min and then cell debris was collected by centrifugation at 12,000×g at 4 C for 15 min. Supernatant was collected, and protein levels quantified using BCA assay (Thermo Fisher Scientific). For western blot 3ug of protein was used for western blot analysis. Prior to western blot equal volume of 2×SDS loading buffer [125 mM Tris-HCl pH 6.8, 20% glycerol, 5% SDS, and 0.2 M DTT] was added to 3ug of protein lysate and briefly boiled at 95° C. for 5 min before loading on a 12% Tris-Glycine SDS-PAGE gel (Thermo Fisher Scientific) and run for app. 1.5 hr at 125 V. The proteins were transferred to a PVDF membrane (BioRad) by wet-blotting for 2 hours at 4° C. at 30 V. Subsequently, the membrane was pre-blocked for 1 hr at RT with 10% skim milk, followed by 1 hr incubation with primary antibody and 1 hr with secondary antibody. After each antibody incubation, the membrane was rinsed 3×5 min in 1×PBS+0.05% Tween20 and 1×5 min wash with 1×PBS. The protein bands were developed using SuperSignal West Femto Maximum Sensitivity Substrate kit (Thermo Fisher Scientific) and ChemiDoc XRS+(Biorad).

### Antibodies Used:

ICOS-L (1:500; ab233151, Abcam)
Renilla (1:2,000; ab185925, Abcam)
eGFP (1:5,000; ab290, Abcam)
FLAG (1:10,000; F3165, Sigma-Aldrich)
Beta-Actin (1:20,000; A5441, Sigma-Aldrich)
Rabbit Secondary (1:10,000; ab6721, Abcam)
Mouse Secondary (1:10,000; ab90723, Abcam)

### Example 1: CircRNA Expression Vector Design

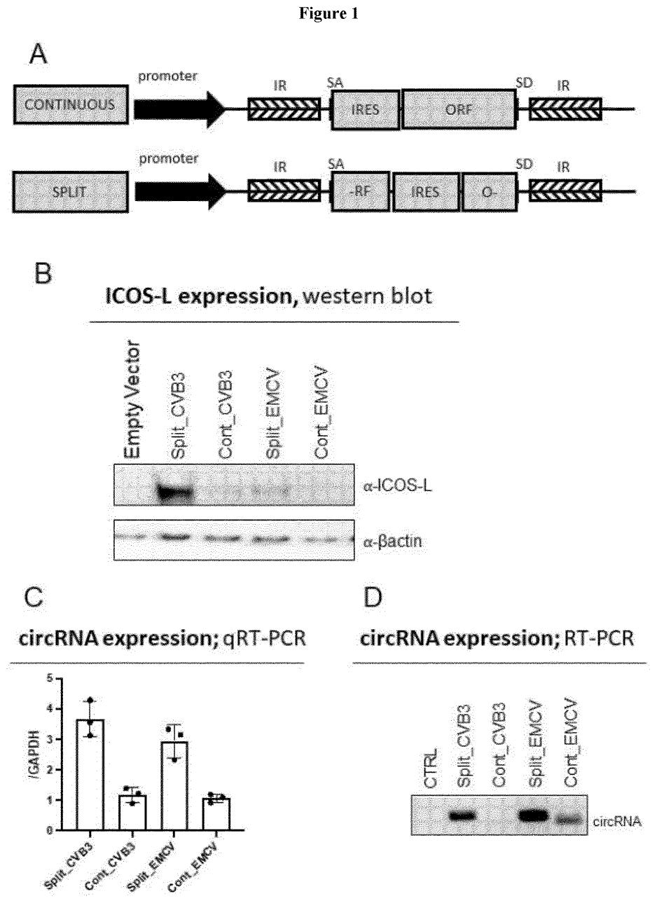

Several vectors comprising circRNA expression cassettes were designed to encode different transgenes from a circular RNA transcript using different IRES-ORF positions (see FIG. 1A). All expression cassettes share a common promoter (CMV) and are flanked by inverted repeat elements (IR-HIPK3) to promote backsplicing and circRNA production. Flanking regions of HIPK3 were utilized in the design of the expression vectors, as the HIPK3 gene encodes one of the highest endogenously expressed circRNAs across multiple human cell and tissue types. Moreover, the circRNA exon is also flanked by splice sites to facilitate backsplicing. Other features such as branch point sequences and polypyrimidine tract sequences are involved to ensure efficient splicing, and these features were taken from the immediate flanking regions of circHIPK3. The open reading frames (ORFs) (coding region of the circRNA) were designed by two different strategies; one where the ORF unit is continuous downstream of the IRES and the other where the ORF is split to flank the IRES element (FIG. 1A). For generation of the split ORF design, ideally a CAGG or AAGG motif located centrally within the ORF is to be identified and the ORF should be split between the two GG nucleotides (where one portion of the ORF ends in CAG and the other begins in G). The rationale of the design is based on the human consensus sequence of splice sites, splice donor (SD) splice site and splice acceptor (SA) splice site, recognized by endogenous splicing machinery. In endogenous splicing the splice donor is joined to the splice acceptor, and the CAGG motif shows the highest level of conservation at these sites. The IRES element is then inserted between the split ORF so that C-terminal portion of the gene is placed upstream of the IRES and the N-terminal downstream (FIG. 1A). Thereby the effects of specific features, particularly choice of IRES and positioning of IRES, on circRNA production are examined.

Natural circRNAs lack 5′cap and 3′ polyA tails necessary for cap-dependent translation and are thus believed to be of non-coding nature. However, it has been shown that introduction 5 of an IRES element within a circRNA molecule can induce translation of the circRNA through cap-independent mechanisms. Thus, the protein coding potential of circRNAs containing an IRES element derived from the CVB3 or EMCV was examined. CircRNA expression vectors encoding ICOS-L were tested using both a continuous and split ORF design (see Table 3 below for ICOS-L split ORF), as described above, containing either a CVB3 or EMCV IRES. ICOS-L protein level in A375 cells transfected with plasmids encoding circICOSL as a continuous or split design containing either the EMCV or CVB3 IRES was examined 48 hours post-transfection. Western blot analysis showed that the CVB3 IRES stimulated superior protein production compared to the EMCV IRES (FIG. 1i). Interestingly, the split design in combination with the CVB3 IRES induced significantly higher levels of ICOS-L protein expression compared to the continuous ORF (FIG. 1i). Furthermore, the continuous ORF design in combination with the EMCV TRES showed no detectable protein output. Additionally, both RT-PCR (FIG. 1C) and qRT-PCR (FIG. 1D) analysis showed that circRNA levels were significantly higher from the split ORF design compared to the continuous ORF design. This suggests that positioning of the IRES within the circRNA expression cassette has a large impact on circRNA production.

### Example 2: Distancing of the IRES to the Backsplicing Sites Affects circRNA Expression

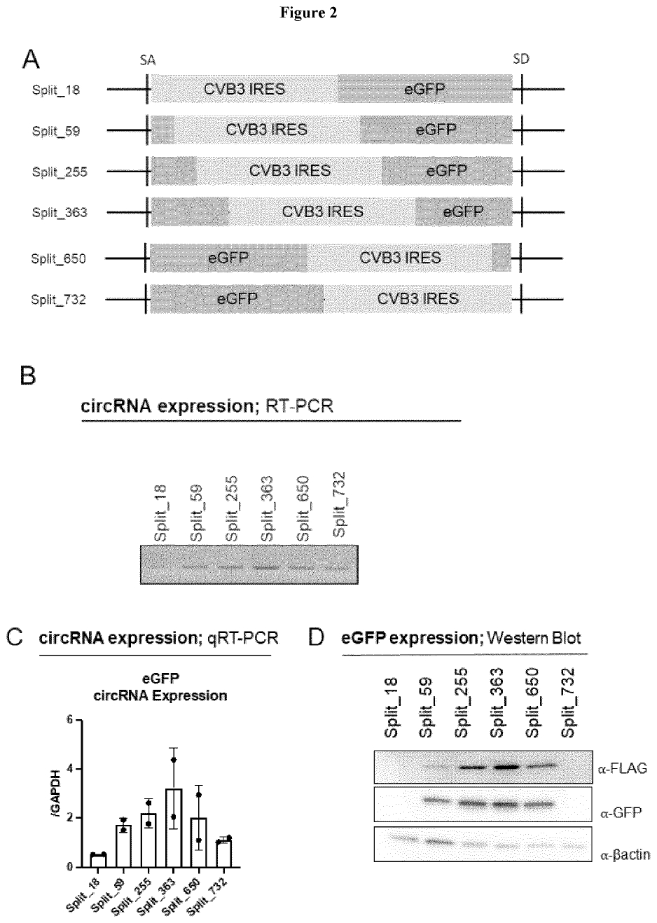

To further study the impact of the split ORF design on circRNA production, a circEGFP expression vector was generated. The expression vector contained all elements described in example 1 above. The eGFP ORF was split at several positions to alter the distance of the IRES (here CVB3) from the flanking splice sites. The circEGFP designs were labelled based on the distance of the IRES from the upstream SA site (i.e. first backsplicing site), e.g. for Split 59 the IRES is inserted 59 nt downstream of the SA (FIG. 2A; Table 1).

A375 cells were transfected with plasmids encoding different split ORF designs of circEGFP. RNA and protein levels were examined 48 hours post-transfection. CircRNA expression from each construct was confirmed by RT-PCR (FIG. 2B), using divergent primers to amplify only circular RNA transcripts. Quantitative RNA and protein expression analysis by qRT-PCR (FIG. 2C) and western blot (FIG. 2D) respectively, demonstrated a negative correlation between proximity of the IRES to the splice sites and circRNA expression. Splitting the ORF near 5′ end (Split 18 & Split 59) or the 3′ end (Split 650 & Split 732) showed reduced circRNA expression at both RNA & protein level compared to a more central split (Split_255 & Split 363) (FIG. 2C-D). This suggests that a distance of ~-250 nt between the IRES and the splice sites facilitates the highest level of circRNA expression. However, at distances ~59-94 nt between the IRES and the splice sites circRNA production is still detectable at a proportionately higher level than when compared to a distance of ~13-18 nt.

TABLE 1
Nucleotide distance between IRES element and upstream splice
acceptor (SA) or downstream splice donor (SD) for each
circEGFP split circRNA expression cassette design.

| | DISTANCE | DISTANCE |
|---|---|---|
| | BETWEEN | BETWEEN |
| | IRES & SA | IRES & SD |
| | (NT) | (NT) |
| SPLIT_18 | 18 | 726 |
| (SEQ ID NO: 006) | | |
| SPLIT_59 | 59 | 685 |
| (SEQ ID NO: 007) | | |
| SPLIT_255 | 255 | 489 |
| (SEQ ID NO: 008) | | |
| SPLIT_363 | 363 | 381 |
| (SEQ ID NO: 009) | | |
| SPLIT_650 | 650 | 94 |
| (SEQ ID NO: 010) | | |
| SPLIT_732 | 732 | 12 |
| (SEQ ID NO: 011) | | |

### Example 3: Splitting of the IRES

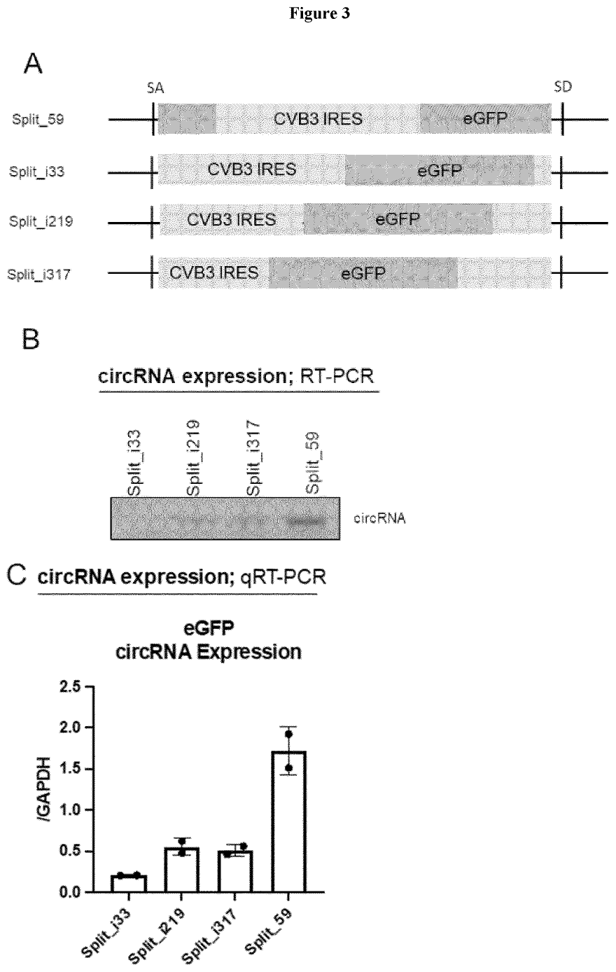

In addition, the inventors examined if splitting the IRES element itself would impact circRNA expression. The expression vector contained all elements described in example 1 above. The IRES element was split at different positions at a CAGG/AAGG motif to allow for splicing and circularization of the circRNA without introducing additional nucleotides in the IRES sequence. In this experiment the continuous eGFP ORF is inserted within the split IRES element and the IRES fragments would immediately flank the backsplicing sites involved in circRNA production (FIG. 3A). The IRES split designs were named based on the distance from the beginning of the IRES (the first nucleotide encoding the IRES) to the SD. The following 20 expression constructs were tested: Split_i33 (SEQ ID NO: 012), Split_i219 (SEQ ID NO: 013) and Split_i317 (SEQ ID NO: 014). A375 cells were transfected with plasmids encoding different split IRES designs of circEGFP. circRNA expression was assessed 48 hours post-transfection by RT-PCR (FIG. 3B) and qRT-PCR (FIG. 3C). Western blotting was conducted to assess eGFP protein expression (FIG. 3D). Splitting of the IRES showed significantly diminished circRNA expression compared to a ORF split design (see FIG. 3B-D). In fact, the IRES-split design consistently showed the lowest level of circRNA expression on both RNA and protein level. Thus, splitting the IRES element and insertion of an internal continuous ORF does not appear to be a suitable system for high-yield circRNA expression.

### Example 4: Proximity of the IRES to Flanking Backsplicing Sites Affects circRNA Expression

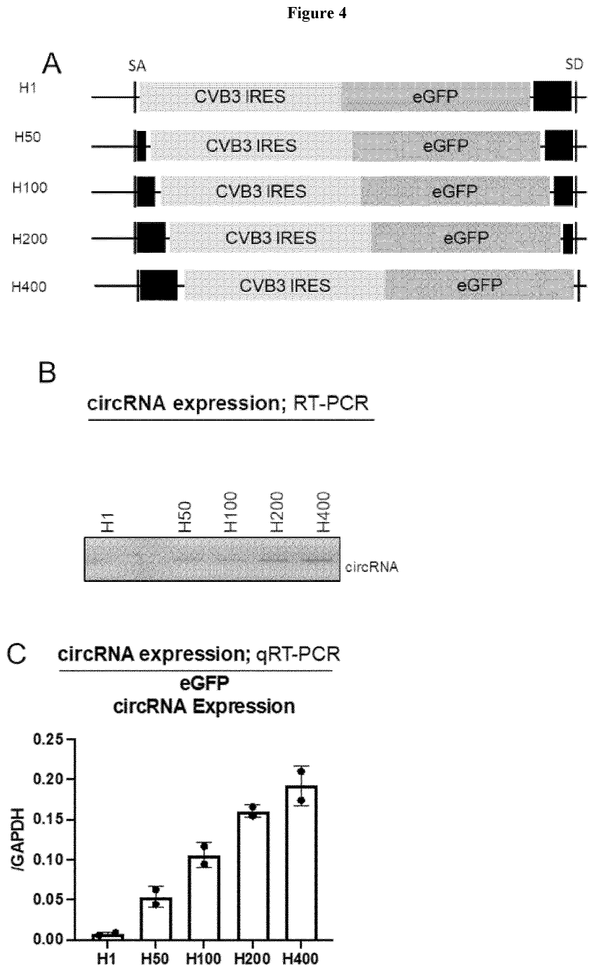

To further examine the impact of IRES positioning in relation to the backsplicing sites on circRNA expression, an eGFP circRNA expression vector was generated based on the circONCOS backbone, i.e. containing identical flanking sequences and splice sites. Here, the relationship between the proximity of the IRES to the splice sites was examined. CircRNA expression cassettes were generated, where the continuous eGFP ORF was inserted downstream of the CVB3 IRES element. Additionally, non-coding ‘spacer’ sequences derived from HIPK3 exon 2 were inserted into the flanking regions of the IRES-ORF cassette (FIG. 4A). To ensure that each of the encoded circRNAs were of identical size, 403 nt of spacer sequence were added to each design, however the amount of spacer sequence inserted upstream and downstream of the IRES-ORF varied between each circRNA design (FIG. 4A). The designs were named based on the size of the HIPK3 stuffer sequence inserted upstream of the IRES, i.e. circH1 had 1 nt inserted upstream of the IRES and 402 nt inserted downstream of the ORF (Table 2). A375 cells were transfected with plasmids encoding circEGFP containing different HIPK3 derived spacer sequences upstream flanking the IRES/ORF. Analysis of circRNA expression by both RT-PCR (FIG. 4B) and qRT-PCR (FIG. 4C) clearly demonstrate a relationship between distance of the IRES from the upstream SA and the level of circRNA expression, in which increased distance between IRES from the SA positively correlate with circRNA expression. In other words, circH1, circH50 and circH100 show significantly decreased levels of circRNA expression compared to circH200 and circH400 (FIG. 4C). RT-PCR showed that the level of circRNA expression is similar between circH200 and circH400. Overall, the data suggests that positioning the IRES distally from the SA increases circRNA yield.

TABLE 2
Nucleotide distance between IRES element and
upstream splice acceptor (SA) or downstream splice
donor (SD) for each circEGFP_HIPK3 spacer
circRNA expression cassette design.

| | DISTANCE | DISTANCE |
|---|---|---|
| | BETWEEN | BETWEEN |
| | IRES & SA | IRES & SD |
| | (NT) | (NT) |
| H1 | 1 | 1146 |
| (SEQ ID NO: 015) | | |
| H50 | 50 | 1097 |
| (SEQ ID NO: 016) | | |
| H100 | 100 | 1047 |
| (SEQ ID NO: 017) | | |
| H200 | 200 | 947 |
| (SEQ ID NO: 018) | | |
| H400 | 400 | 747 |
| (SEQ ID NO: 019) | | |

### Example 5: Split ORF Design Provides Improved circRNA Yield and Higher Protein Translation

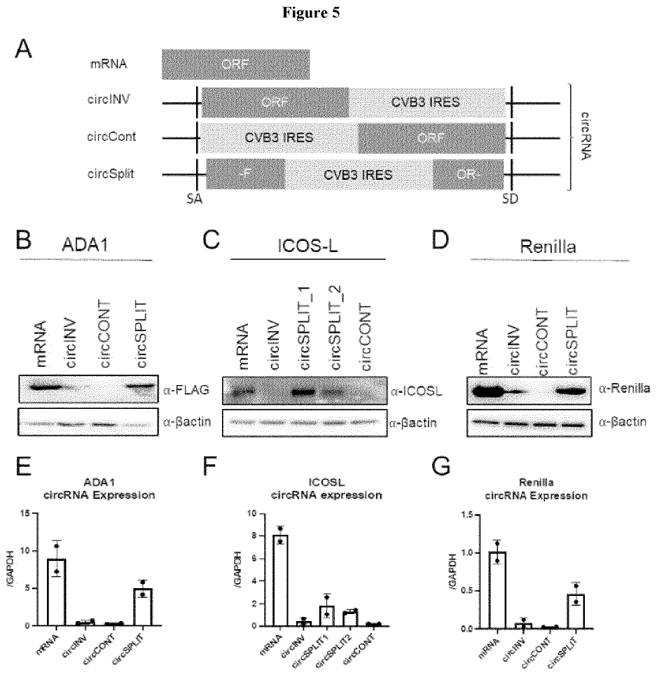

To generalize our observations that the split ORF design is the superior circRNA expression design independent of a particular ORF, circRNA expression from constructs encoding different transgenes was examined. Three additional transgenes encoding the co-stimulatory molecule ICOS-L, adenosine deaminase ADA, and the reporter gene Renilla luciferase were examined. Three circRNA designs were tested, where the continuous transgene ORF was placed upstream (circInv) or downstream (circCont) of the IRES, in addition to a centrally split ORF design (circSplit), wherein at least 30% of the ORF was placed between the 3′ end of the IRES and the SD (FIG. 5A; Table 3). For ICOS-L additional split designs were tested. For ICOS-L both cassettes were split at the same motif, but version 2 contained a short spacer sequence upstream of the start codon. Additionally, RNA and protein levels were compared to the corresponding linear mRNA.

A375 cells were transfected with plasmids encoding circular and linear versions of ICOS-L (SEQ ID NO: 21), Renilla (SEQ ID NO: 22) and FLAG-tagged ADA (SEQ ID NO: 20). Western blot analysis of protein expression from the circRNA cassettes show that the expression levels from the circSplit design are significantly higher than those derived from either the circInv or circCont design (FIG. 5B-D). Furthermore, across all three transgenes, qRT-PCR analysis showed the circSplit design gave superior circRNA expression compared to the circInv or circCont design (FIG. 5E-G). These observations support the hypothesis that splitting the ORF centrally and placing the CVB3 IRES internally within the ORF gives rise to superior circRNA expression at both RNA and protein levels.

TABLE 3
Nucleotide distance between IRES element and
upstream splice acceptor (SA) or downstream splice
donor (SD) for each circEGFP_HIPK3 spacer
circRNA expression cassette design.

| | DISTANCE | DISTANCE |
|---|---|---|
| | BETWEEN | BETWEEN |
| | IRES & SA | IRES & SD |
| | (NT) | (NT) |
| SPLIT_ADA | 497 | 587 |
| SPLIT_ICOSL | 591 | 318 |
| SPLIT_RENILLA | 72 | 864 |

Additionally, we observe for each of the transgene a similar (FIG. 5B ADA; FIG. 5D Renilla) or superior (FIG. 5C ICOS-L) level of protein expression from the circSplit design compared to their linear mRNA counterparts. Strikingly, these comparable/superior protein levels are observed regardless of the significantly lower RNA levels of the circRNA compared to the corresponding mRNA (FIG. 5E-G). This suggests that the CVB3 IRES in combination with a split ORF design is a powerful tool for transgene protein expression, that can potentially outcompete conventional linear mRNA expression cassettes even at early timepoints when mRNA expression is highest.

### Example 6: Positioning of IRES within in circRNA Cassette Impacts circRNA Expression Independent of Expressed Gene or Used IRES

As demonstrated in the above examples, positioning of the IRES within the circRNA dramatically enhances circRNA expression. These findings are equally achieved with a different gene, ICOSL. Similar to the above experiments, the ICOSL ORF was split at different positions and the IRES was placed between the stop codon (upstream of IRES) and start codon (downstream of IRES), modulating the distance between the IRES and splice acceptor (SA) and splice donor (SD) sites. The exact split positions are reported in Table 4.

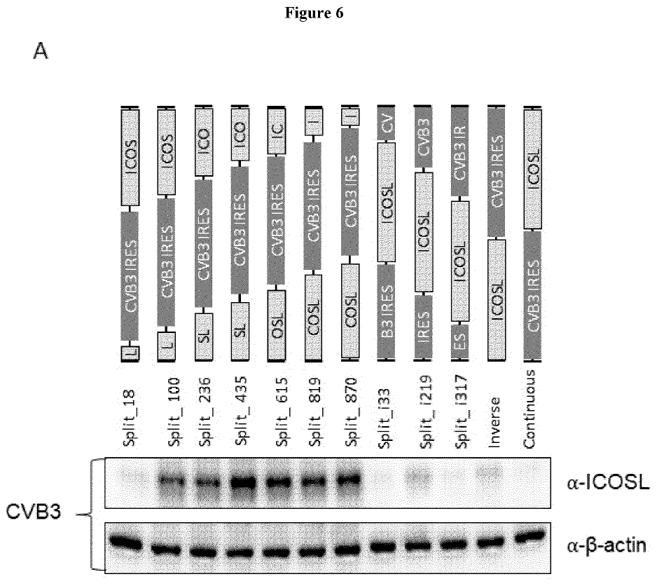

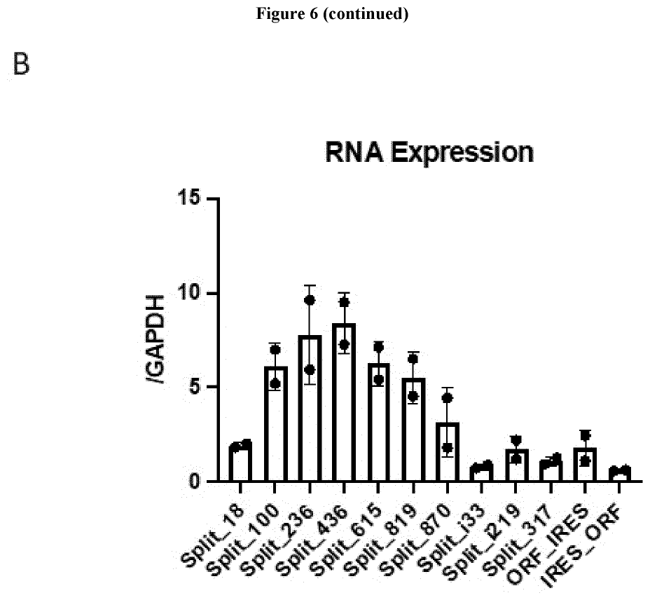

Briefly, A375 cells were transfected with plasmids encoding different split ORF designs of circICOSL. RNA and protein levels were examined 48 hours post-transfection. Protein and circRNA expression analysis was conducted by western blot (FIG. 6A) and by qRT-PCR (FIG. 6B) respectively. As previously demonstrated, there was a negative correlation between proximity of the IRES to the splice sites and circRNA expression. Splitting the ORF near the 5′ end (Split_18) or the 3′ end (Split_870) showed reduced circRNA expression at both an RNA and protein level compared to a more central split (Split_236 & Split_436). Additionally, as previously reported, splitting the IRES (i33, i219, i317) reduces circRNA expression on protein and RNA level (FIG. 6A-B). Overall, these results further support the observation that placing the IRES centrally within a split ORF, resulting in similar distances between the IRES to either SA or SD, yields the highest protein expression levels.

TABLE 4
Nucleotide distance between IRES element and upstream splice
acceptor (SA) or downstream splice donor (SD) for each
circICOSL split circRNA expression cassette design.

| | Distance | Distance |
|---|---|---|
| | between | between |
| | IRES & SA | IRES & SD |
| | (nt) | (nt) |
| Split_18 | 18 | 915 |
| Split_59 | 100 | 833 |
| Split_255 | 236 | 697 |
| Split_363 | 436 | 497 |
| Split_650 | 615 | 318 |
| Split_819 | 819 | 114 |
| Split_870 | 870 | 63 |
| | Distance | Distance |
| | between | between |
| | ORF & SA | ORF & SD |
| IRES SPLIT | (nt) | (nt) |
| Split_i33 | 708 | 33 |
| Split_i219 | 522 | 219 |
| Split_i317 | 424 | 317 |
| | Distance | Distance |
| | between | between |
| | IRES & SA | IRES & SD |
| Continuous ORF | (nt) | (nt) |
| IRES_ORF | 0 | 933 |
| ORF_IRES | 933 | 0 |

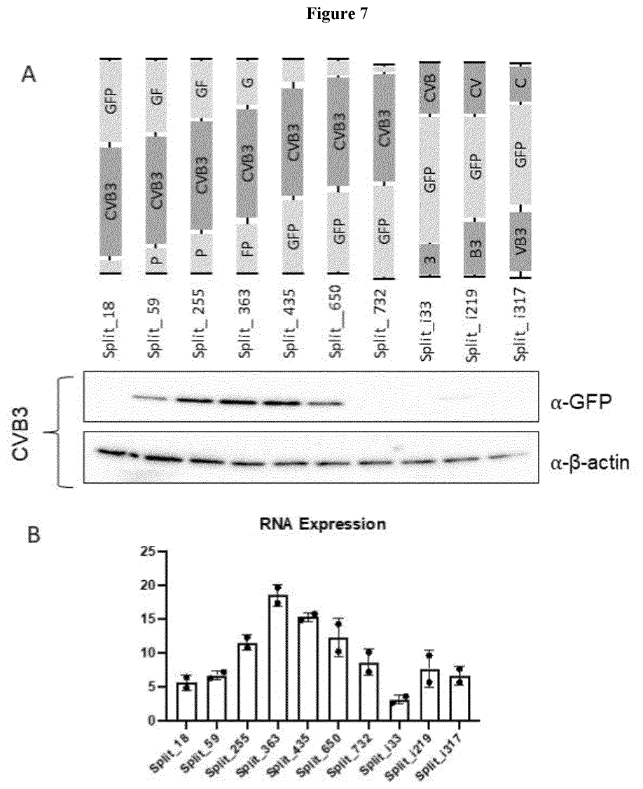

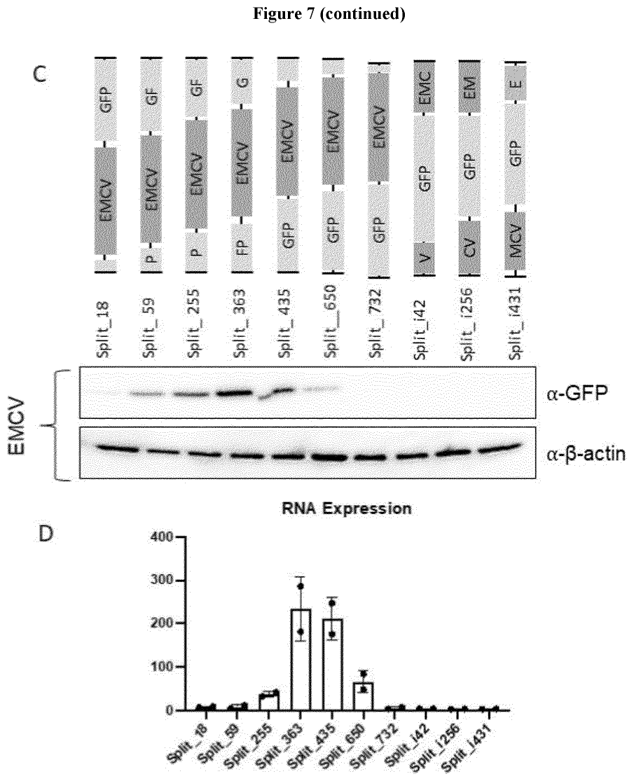

Further supporting this observation, when the CVB3 IRES (741nt; Group I IRES) used in the previous examples was replaced with the EMCV IRES (566nt; Group II IRES) in the eGFP encoding circRNA vectors (Table 5), a similar circEGFP expression pattern was observed. A375 cells were transfected with plasmids encoding different split ORF designs of circEGFP with either a CVB3 (FIG. 7A-B) or EMCV IRES (FIG. 7C-D). Protein and RNA levels were examined 48 hours post-transfection. Protein and circRNA expression analysis was conducted by western blot (FIG. 7A&C) and by qRT-PCR (FIG. 7B&D) respectively. This supported that the ORF-IRES positioning is important for high yield circRNA expression independent of the IRES element used. A similar circRNA expression pattern for both the CVB3 and EMCV circRNA designs was observed, where a more central split (Split_363 and Split_436) gives the highest level of circRNA expression on both protein and RNA level. Additionally, similar to the results in the above examples, splitting the ORF nearer to 5′ and 3′ ends negatively impacts circRNA biogenesis. Furthermore, splitting the EMCV IRES and placing the ORF within this region also inhibits high yield circRNA expression (FIG. 7C-D), similar to what was observed for the CVB3 IRES split designs (FIG. 7A-B).

TABLE 5
Nucleotide distance between IRES or eGFP_ORF and upstream
splice acceptor (SA) or downstream splice donor (SD) for
each circEGFP split circRNA expression cassette design.

| | Distance | Distance |
|---|---|---|
| | between | between |
| | IRES & SA | IRES & SD |
| | (nt) | (nt) |
| Split_18 | 18 | 726 |
| Split_59 | 59 | 685 |
| Split_255 | 255 | 489 |
| Split_363 | 363 | 381 |
| Split_435 | 435 | 309 |
| Split_650 | 650 | 94 |
| Split_732 | 732 | 12 |
| | Distance | Distance |
| | between | between |
| | ORF & SA | ORF & SD |
| CVB3 Split | (nt) | (nt) |
| Split_i33 | 708 | 33 |
| Split_i219 | 522 | 219 |
| Split_i317 | 424 | 317 |
| | Distance | Distance |
| | between | between |
| | ORF & SA | ORF & SD |
| EMCV Split | (nt) | (nt) |
| Split_i42 | 524 | 42 |
| Split_i256 | 310 | 256 |
| Split_i431 | 135 | 431 |
| | Distance | Distance |
| | between | between |
| | IRES & SA | IRES & SD |
| Continuous ORF | (nt) | (nt) |
| IRES_ORF | 0 | 933 |
| ORF_IRES | 933 | 0 |

Overall, the data supports the hypothesis that positioning the IRES centrally within the split ORF design gives the highest level of circRNA expression. The inventors hypothesize that this increased expression is a consequence of increasing the distance of the IRES from the flanking splice sites required for backsplicing, as a central split allows for the maximum distance of the IRES from either splice site.

1. A nucleic acid molecule encoding a circular RNA (circRNA), wherein the nucleic acid molecule comprises:
A) an expression cassette comprising:
(i) a circRNA expression cassette comprising:
(a) a nucleic acid comprising or consisting of a continuous or split open reading frame (ORF) encoding at least one protein, and
(b) an internal ribosome entry site (IRES) operably linked to the ORF to direct translation of the ORF;
(ii) a first backsplicing site that is positioned 5′ of the IRES; and
(iii) a second backsplicing site that is positioned 3′ of the IRES;
and optionally
B) a first inverted repeat (IR) element positioned 5′ of the first backsplicing site and a second IR element positioned 3′ of the second backsplicing site; and/or
a promoter operably linked to the expression cassette to direct expression of the expression cassette;
wherein the IRES is positioned within the circRNA expression cassette in that:
(I) the 5′ end of the IRES and the 3′ end of the first backsplicing site are separated by at least 50 nucleotides, preferably by at least 200 more preferably by at least 300 nucleotides, and
(II) the 3′ end of the IRES and the 5′ end of the second backsplicing site are separated by at least 300 nucleotides, preferably by at least 350 nucleotides.

2. The nucleic acid molecule according to claim 1, wherein the split ORF comprises two parts and the first part of the split ORF comprises a STOP codon and is positioned 5′ of the IRES and the second part of the split ORF comprises a START codon and is positioned 3′ of the IRES.

3. The nucleic acid molecule according to claim 1, wherein the first backsplicing site is comprising two half-sites, wherein
the first and the second half-site are positioned 5′ of the circRNA expression cassette; or
the first half-site is positioned 5′ of the circRNA expression cassette and the second half-site is part of the circRNA expression cassette, preferably being part of the ORF in the case of a split ORF;
and, wherein the second backsplicing site is comprising two half-sites, wherein
the first and the second half-site are positioned 3′ of the circRNA expression cassette; or
the first half-site is part of the circRNA expression cassette, preferably being part of the ORF in the case of a split ORF, and the second half-site is positioned 3′ of the circRNA expression cassette.

4. The nucleic acid molecule according to claim 1, wherein
the first half site of the first backsplicing site comprises or consists of the nucleotides AG; and/or
the second half site of the second backsplicing site comprises or consists of the nucleotides GT.

5. The nucleic acid molecule according to claim 1, wherein the IRES is a viral IRES,
preferably of a virus selected from the group of Coxsackievirus (CVB), preferably CVB3, and encephalomyocarditis virus (EMCV); or
preferably selected from the group of: Adenoviridae, Arenaviridae, Birnaviridae, Chrysoviridae, Coronaviridae, Dicistroviridae, Filoviridae, Flaviviridae, Hepadnaviridae, Herpesviridae, Hypoviridae, Iflaviridae, Luteoviridae, Orthomyxoviridae, Papillomaviridae, Paramyxoviridae, Parvoviridae, Picornaviridae, Pneumoviridae, Polyomaviridae, Potyviridae, Reoviridae, Retroviridae, Rhabdoviridae, Secoviridae, Tombusviridae, Totiviridae, Virgaviridae; or
preferably a variant of the viral IRES, which has a nucleotide sequence that is at least 90% identical to the nucleotide sequence of the viral IRES.

6. The nucleic acid molecule according to claim 1, wherein the first and the second IR element comprise a sequence being at least 80%, preferably at least 90%, more preferably at least 95% identical to the reverse complementary sequence of each other.

7. The nucleic acid molecule according to claim 1, wherein the IR elements are selected from the group consisting of IR elements derived from the genes of: AC004076.9, ACYP2, AMD1, ARHGAP10, ARHGAP12, ARHGEF12, ARHGEF28, ASAP1, ASH1L, ASXL1, ATXN2, BMPR2, BRWD1, BTBD10, CBFA2T2, CCDC126, CCDC134, CCDC66, CCDC7, CCDC9, CCNB1, CDK13, CFLAR, CHD9, CLIP2, CLNS1A, CNN2, COA1, CORO1C, CREBBP, CRKL, CTB-43P18.3,SNHG4, DCUN1D4, DEK, DHRS3, DLG1, DOPEY2, DYNC1H1, ELF2, EMC2, EPHB4, EPS15, ERC1, ETFA, EXOSC1, FAM13B, FARSA, FBXO7, FGD4, FGD6, FKBP3, FKBP8, FNTA, FOXK2, GAPVD1, GBAS, GDI2, GLIS2, GLS, GON4L, GRHPR, HERC1, HIPK3, HNRNPM, HOOK3, HP1BP3, HPS5, HTT, HUWE1, IARS, ILKAP, IQGAP1, KDM1A, KIAA0368, KIAA1429, KIAA1841, KLHL8, KMT2C, LMBR1, LRCH3, LZIC, MAP3K1, MARK4, MBOAT2, MCU, MED13L, METTL3, MGA, MGEA5, MITD1, MORC3, MRPS35, MYO9B, NCOA2, NFAT5, NFATC3, NFX1, NUDC, NUP54, PAFAH1B2, PAIP2, PCMT1, PDCD11, PDE8A, PDS5A, PHC3, PHLDB2, PLEKHM1, PLEKHM3, PLOD2, PMS1, PNN, POLR2A, POMT1, PPP6R2, PROSC, PRRC2B, PSEN1, PSMA7, PTP4A2, PTPN12, QKI, R3HDM1, RAB6A, RALBP1, RARS, RBM23, RBM33, RBM39, RELL1, REPS1, RERE, RHOBTB3, RLF, RNF19B, SDHAF2, ZRANB1, FAM228B, RPRD1B, RPS6KC1, RSF1, RSRC1, RTN4, RYK, SAFB2, SAMD4A, SCAF8, SCARFI, SCYL2, SDF4, SEC31A, SENP6, SIPA1L1, SKA3, SLC38A1, SLTM, SMARCA5, SMC3, SMO, SNX25, SOBP, SOS2, SPIDR, SPPL3, SRSF4, STAM, STK3, STX6, TERF2, TIMMDC1, TMED2, TMEM138, TMEM165, TMEM181, TNPO1, TNPO3, TOP1, TTC39C, TTLL1, UBA2, UBAP2, UBE2K, UBQLN1, UBR5, UBXN2A, UBXN7, UHRF2, UIMC1, URI1, UTP18, UTRN, VAMP3, VAPB, VMP1, WDR78, XPO1, YTHDF2, YWHAE, YYlAP1, ZBTB46, ZCCHC11, ZCCHC6, ZFAND6, ZFX, ZKSCAN1, ZMYM4, ZNF124, ZNF236, ZNF394, ZNF430, ZNF652, ZNF720, ZNF91], preferably of HIPK3 and ZKSCAN1.

8. The nucleic acid molecule according to claim 1, wherein the promoter is selected from a constitutive, tissue specific or inducible promoter, preferably a viral promoter, preferably selected from the group consisting of the CMV immediate early promoter, the SV40 promoter.

9. The nucleic acid molecule according to claim 1, wherein the circRNA expression cassette further comprises:
(i) one or more non-coding nucleotide sequence(s), if the circRNA expression cassette comprises a continuous ORF then the non-coding nucleotide sequence(s) is(are) preferably positioned at the 5′ end and/or 3′ end of the continuous ORF or if the circRNA expression cassette comprises a split ORF then the non-coding nucleotide sequence(s) is(are) preferably positioned at the 5′ end and/or the 3′ end of the IRES;
(ii) one or more additional ORFs;
(iii) one or more additional IRES;
(iv) an additional coding nucleotide sequence attached to one or more ORF, preferably encoding a sequence that can be used to detect or capture the protein encoded in the one or more ORF.

10. The nucleic acid molecule according to claim 1, wherein the nucleic acid molecule further comprises:
(i) a branch point; and/or
(ii) a polypyrimidine tract

11. The nucleic acid molecule according to claim 1, wherein the ORF encodes a therapeutic protein, therapeutic peptide, antigenic protein or antigenic peptide.

12. A circRNA encoded by the circRNA expression cassette of the nucleic acid molecule of claim 1.

13. A vector comprising the nucleic acid molecule according to claim 1.

14. A host cell comprising the nucleic acid molecule according to claim 1.

15. A pharmaceutical composition comprising the nucleic acid molecule according to claim 1.

16. A method for producing circular mRNA (circRNA), said method comprising:
(i) introducing the nucleic acid molecule of claim 1 in a eukaryotic cell and optionally purifying circRNA from the cell;
or
(ii) contacting the nucleic acid molecule of claim 1 with isolated RNA polymerase II.

17. A method for producing a recombinant protein, said method comprising introducing the nucleic acid molecule of claim 1 into a eukaryotic cell and optionally purifying the recombinant protein encoded by the ORF.


SEQUENCE LISTING (22 sequences)

SEQ ID NO: 1 - DNA, 741 bp, synthetic construct
  source 1..741: mol_type = other DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 2 - DNA, 563 bp, synthetic construct
  source 1..563: mol_type = other DNA
cccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataat

SEQ ID NO: 3 - DNA, 273 bp, synthetic construct
  source 1..273: mol_type = other DNA
tttttttttgagacagagtcttgctctgtctcacaggctgaagtgcagtggtgcaatctcggctcactgcaacctccacctcccaggttcaagcgattctcctccctcagcctcccgagtagctgggaccacaggcatgcaccaccatccccagctaatttttgcattattagtagagttgggatttcttcaccgtgttggccaggctggtcttggactcctgacctcaagtgatccaactgcctcagcctctcaaagtgctaggattacagg

SEQ ID NO: 4 - DNA, 301 bp, synthetic construct
  source 1..301: mol_type = other DNA
gccgggtgcagtggctcatgcctgtaatccctgcacttagggaggctgaggcgggtggatgacctgaggttaggagttcaagaccagcctggccaacatggcgaaacccccatctctactaaaaataacaaaaattagctgggtgtggtggtgggtgtctataatcccagcaacttgggaggctgaggcaggagaatcacttgaacccaggagatggaggttgcagtgagccgagatcatgccattgcactccagcctgggccacaagagcaaaactctgtctcaaaaaaacaaaacaaaa

SEQ ID NO: 5 - DNA, 617 bp, synthetic construct
  source 1..617: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggc

SEQ ID NO: 6 - DNA, 3079 bp, synthetic construct
  source 1..3079: mol_type = other DNA
ctttttttttgagacagagtcttgctctgtctcacaggctgaagtgcagtggtgcaatctcggctcactgcaacctccacctcccaggttcaagcgattctcctccctcagcctcccgagtagctgggaccacaggcatgcaccaccatccccagctaatttttgcattattagtagagttgggatttcttcaccgtgttggccaggctggtcttggactcctgacctcaagtgatccaactgcctcagcctctcaaagtgctaggattacagggatctatacttttcttttgagggaaaatgttggcaccgtttctagggcatattggccatttcagcttctcagtaaatatttgttaagtaattaaatgcacttgattctttattcttagccttttaacgcaatactcagaatagctgaagcaccaattaactgaaatggagatattataaagatagttatcttctccaagggaaaaaatcatcttcatggaaattaattacttttttacaaattgtgaatttgacccttaagagttttcttcctgatatttaaaattgaaaaaaaaattgttgacattaatatttcttctttccttttttttcttttcctttttttttttttttttgcaggacgacgatgacaagtaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggactacaaggtaggtaacaactccatactttttggttgtttattaatgtgaaatttctgctaaatgaaatacttttgtgtgtgtttgtggtagaagagaccacttcagttaaataaggaaatcaagagaggatcaatttaggttcgttttaaagagattaaaaaaaatcaagacataaaatctacccaagcaggatagaaatctccactgcaaagttccatgccaaagacatctggttatttttatttttaatggaagacttgaaggaatgataggtgattaataatgatcaaacagaagtctttaaatgttggaaagtatttacattaatctttgtatatatcattgggcattttagcacttgagagaaatagtttattaaagatataatcaatcatatgtaactgaacatttagaaaaattatatacaggtttgagtagcccttatctgaaacttttggggccagaagtgttttggattccagatttttccggattttggaatatttgcactgccaactagttaagcacccccaaatttgaaaattcgtttcctttgagtgtcatgtcaatgcccaaaaagtttcagatatttggatttgagatgctcaacctgtataaggattcagaaagttattctgattaatgattttaagattcagatatacagccgggtgcagtggctcatgcctgtaatccctgcacttagggaggctgaggcgggtggatgacctgaggttaggagttcaagaccagcctggccaacatggcgaaacccccatctctactaaaaataacaaaaattagctgggtgtggtggtgggtgtctataatcccagcaacttgggaggctgaggcaggagaatcacttgaacccaggagatggaggttgcagtgagccgagatcatgccattgcactccagcctgggccacaagagcaaaactctgtctcaaaaaaacaaaacaaaa

SEQ ID NO: 7 - DNA, 3079 bp, synthetic construct
  source 1..3079: mol_type = other DNA
ctttttttttgagacagagtcttgctctgtctcacaggctgaagtgcagtggtgcaatctcggctcactgcaacctccacctcccaggttcaagcgattctcctccctcagcctcccgagtagctgggaccacaggcatgcaccaccatccccagctaatttttgcattattagtagagttgggatttcttcaccgtgttggccaggctggtcttggactcctgacctcaagtgatccaactgcctcagcctctcaaagtgctaggattacagggatctatacttttcttttgagggaaaatgttggcaccgtttctagggcatattggccatttcagcttctcagtaaatatttgttaagtaattaaatgcacttgattctttattcttagccttttaacgcaatactcagaatagctgaagcaccaattaactgaaatggagatattataaagatagttatcttctccaagggaaaaaatcatcttcatggaaattaattacttttttacaaattgtgaatttgacccttaagagttttcttcctgatatttaaaattgaaaaaaaaattgttgacattaatatttcttctttccttttttttcttttcctttttttttttttttttgcagggatcactctcggcatggacgagctgtacaaggactacaaggacgacgatgacaagtaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgcaggtaggtaacaactccatactttttggttgtttattaatgtgaaatttctgctaaatgaaatacttttgtgtgtgtttgtggtagaagagaccacttcagttaaataaggaaatcaagagaggatcaatttaggttcgttttaaagagattaaaaaaaatcaagacataaaatctacccaagcaggatagaaatctccactgcaaagttccatgccaaagacatctggttatttttatttttaatggaagacttgaaggaatgataggtgattaataatgatcaaacagaagtctttaaatgttggaaagtatttacattaatctttgtatatatcattgggcattttagcacttgagagaaatagtttattaaagatataatcaatcatatgtaactgaacatttagaaaaattatatacaggtttgagtagcccttatctgaaacttttggggccagaagtgttttggattccagatttttccggattttggaatatttgcactgccaactagttaagcacccccaaatttgaaaattcgtttcctttgagtgtcatgtcaatgcccaaaaagtttcagatatttggatttgagatgctcaacctgtataaggattcagaaagttattctgattaatgattttaagattcagatatacagccgggtgcagtggctcatgcctgtaatccctgcacttagggaggctgaggcgggtggatgacctgaggttaggagttcaagaccagcctggccaacatggcgaaacccccatctctactaaaaataacaaaaattagctgggtgtggtggtgggtgtctataatcccagcaacttgggaggctgaggcaggagaatcacttgaacccaggagatggaggttgcagtgagccgagatcatgccattgcactccagcctgggccacaagagcaaaactctgtctcaaaaaaacaaaacaaaa

SEQ ID NO: 8 - DNA, 3079 bp, synthetic construct
  source 1..3079: mol_type = other DNA
ctttttttttgagacagagtcttgctctgtctcacaggctgaagtgcagtggtgcaatctcggctcactgcaacctccacctcccaggttcaagcgattctcctccctcagcctcccgagtagctgggaccacaggcatgcaccaccatccccagctaatttttgcattattagtagagttgggatttcttcaccgtgttggccaggctggtcttggactcctgacctcaagtgatccaactgcctcagcctctcaaagtgctaggattacagggatctatacttttcttttgagggaaaatgttggcaccgtttctagggcatattggccatttcagcttctcagtaaatatttgttaagtaattaaatgcacttgattctttattcttagccttttaacgcaatactcagaatagctgaagcaccaattaactgaaatggagatattataaagatagttatcttctccaagggaaaaaatcatcttcatggaaattaattacttttttacaaattgtgaatttgacccttaagagttttcttcctgatatttaaaattgaaaaaaaaattgttgacattaatatttcttctttccttttttttcttttcctttttttttttttttttgcaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggactacaaggacgacgatgacaagtaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtaggtaacaactccatactttttggttgtttattaatgtgaaatttctgctaaatgaaatacttttgtgtgtgtttgtggtagaagagaccacttcagttaaataaggaaatcaagagaggatcaatttaggttcgttttaaagagattaaaaaaaatcaagacataaaatctacccaagcaggatagaaatctccactgcaaagttccatgccaaagacatctggttatttttatttttaatggaagacttgaaggaatgataggtgattaataatgatcaaacagaagtctttaaatgttggaaagtatttacattaatctttgtatatatcattgggcattttagcacttgagagaaatagtttattaaagatataatcaatcatatgtaactgaacatttagaaaaattatatacaggtttgagtagcccttatctgaaacttttggggccagaagtgttttggattccagatttttccggattttggaatatttgcactgccaactagttaagcacccccaaatttgaaaattcgtttcctttgagtgtcatgtcaatgcccaaaaagtttcagatatttggatttgagatgctcaacctgtataaggattcagaaagttattctgattaatgattttaagattcagatatacagccgggtgcagtggctcatgcctgtaatccctgcacttagggaggctgaggcgggtggatgacctgaggttaggagttcaagaccagcctggccaacatggcgaaacccccatctctactaaaaataacaaaaattagctgggtgtggtggtgggtgtctataatcccagcaacttgggaggctgaggcaggagaatcacttgaacccaggagatggaggttgcagtgagccgagatcatgccattgcactccagcctgggccacaagagcaaaactctgtctcaaaaaaacaaaacaaaa

SEQ ID NO: 9 - DNA, 3079 bp, synthetic construct
  source 1..3079: mol_type = other DNA
ctttttttttgagacagagtcttgctctgtctcacaggctgaagtgcagtggtgcaatctcggctcactgcaacctccacctcccaggttcaagcgattctcctccctcagcctcccgagtagctgggaccacaggcatgcaccaccatccccagctaatttttgcattattagtagagttgggatttcttcaccgtgttggccaggctggtcttggactcctgacctcaagtgatccaactgcctcagcctctcaaagtgctaggattacagggatctatacttttcttttgagggaaaatgttggcaccgtttctagggcatattggccatttcagcttctcagtaaatatttgttaagtaattaaatgcacttgattctttattcttagccttttaacgcaatactcagaatagctgaagcaccaattaactgaaatggagatattataaagatagttatcttctccaagggaaaaaatcatcttcatggaaattaattacttttttacaaattgtgaatttgacccttaagagttttcttcctgatatttaaaattgaaaaaaaaattgttgacattaatatttcttctttccttttttttcttttcctttttttttttttttttgcagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggactacaaggacgacgatgacaagtaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaaggtaggtaacaactccatactttttggttgtttattaatgtgaaatttctgctaaatgaaatacttttgtgtgtgtttgtggtagaagagaccacttcagttaaataaggaaatcaagagaggatcaatttaggttcgttttaaagagattaaaaaaaatcaagacataaaatctacccaagcaggatagaaatctccactgcaaagttccatgccaaagacatctggttatttttatttttaatggaagacttgaaggaatgataggtgattaataatgatcaaacagaagtctttaaatgttggaaagtatttacattaatctttgtatatatcattgggcattttagcacttgagagaaatagtttattaaagatataatcaatcatatgtaactgaacatttagaaaaattatatacaggtttgagtagcccttatctgaaacttttggggccagaagtgttttggattccagatttttccggattttggaatatttgcactgccaactagttaagcacccccaaatttgaaaattcgtttcctttgagtgtcatgtcaatgcccaaaaagtttcagatatttggatttgagatgctcaacctgtataaggattcagaaagttattctgattaatgattttaagattcagatatacagccgggtgcagtggctcatgcctgtaatccctgcacttagggaggctgaggcgggtggatgacctgaggttaggagttcaagaccagcctggccaacatggcgaaacccccatctctactaaaaataacaaaaattagctgggtgtggtggtgggtgtctataatcccagcaacttgggaggctgaggcaggagaatcacttgaacccaggagatggaggttgcagtgagccgagatcatgccattgcactccagcctgggccacaagagcaaaactctgtctcaaaaaaacaaaacaaaa

SEQ ID NO: 10 - DNA, 3079 bp, synthetic construct
  source 1..3079: mol_type = other DNA
ctttttttttgagacagagtcttgctctgtctcacaggctgaagtgcagtggtgcaatctcggctcactgcaacctccacctcccaggttcaagcgattctcctccctcagcctcccgagtagctgggaccacaggcatgcaccaccatccccagctaatttttgcattattagtagagttgggatttcttcaccgtgttggccaggctggtcttggactcctgacctcaagtgatccaactgcctcagcctctcaaagtgctaggattacagggatctatacttttcttttgagggaaaatgttggcaccgtttctagggcatattggccatttcagcttctcagtaaatatttgttaagtaattaaatgcacttgattctttattcttagccttttaacgcaatactcagaatagctgaagcaccaattaactgaaatggagatattataaagatagttatcttctccaagggaaaaaatcatcttcatggaaattaattacttttttacaaattgtgaatttgacccttaagagttttcttcctgatatttaaaattgaaaaaaaaattgttgacattaatatttcttctttccttttttttcttttcctttttttttttttttttgcaggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggactacaaggacgacgatgacaagtaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtcaggtaggtaacaactccatactttttggttgtttattaatgtgaaatttctgctaaatgaaatacttttgtgtgtgtttgtggtagaagagaccacttcagttaaataaggaaatcaagagaggatcaatttaggttcgttttaaagagattaaaaaaaatcaagacataaaatctacccaagcaggatagaaatctccactgcaaagttccatgccaaagacatctggttatttttatttttaatggaagacttgaaggaatgataggtgattaataatgatcaaacagaagtctttaaatgttggaaagtatttacattaatctttgtatatatcattgggcattttagcacttgagagaaatagtttattaaagatataatcaatcatatgtaactgaacatttagaaaaattatatacaggtttgagtagcccttatctgaaacttttggggccagaagtgttttggattccagatttttccggattttggaatatttgcactgccaactagttaagcacccccaaatttgaaaattcgtttcctttgagtgtcatgtcaatgcccaaaaagtttcagatatttggatttgagatgctcaacctgtataaggattcagaaagttattctgattaatgattttaagattcagatatacagccgggtgcagtggctcatgcctgtaatccctgcacttagggaggctgaggcgggtggatgacctgaggttaggagttcaagaccagcctggccaacatggcgaaacccccatctctactaaaaataacaaaaattagctgggtgtggtggtgggtgtctataatcccagcaacttgggaggctgaggcaggagaatcacttgaacccaggagatggaggttgcagtgagccgagatcatgccattgcactccagcctgggccacaagagcaaaactctgtctcaaaaaaacaaaacaaaa

SEQ ID NO: 11 - DNA, 3079 bp, synthetic construct
  source 1..3079: mol_type = other DNA
ctttttttttgagacagagtcttgctctgtctcacaggctgaagtgcagtggtgcaatctcggctcactgcaacctccacctcccaggttcaagcgattctcctccctcagcctcccgagtagctgggaccacaggcatgcaccaccatccccagctaatttttgcattattagtagagttgggatttcttcaccgtgttggccaggctggtcttggactcctgacctcaagtgatccaactgcctcagcctctcaaagtgctaggattacagggatctatacttttcttttgagggaaaatgttggcaccgtttctagggcatattggccatttcagcttctcagtaaatatttgttaagtaattaaatgcacttgattctttattcttagccttttaacgcaatactcagaatagctgaagcaccaattaactgaaatggagatattataaagatagttatcttctccaagggaaaaaatcatcttcatggaaattaattacttttttacaaattgtgaatttgacccttaagagttttcttcctgatatttaaaattgaaaaaaaaattgttgacattaatatttcttctttccttttttttcttttcctttttttttttttttttgcagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggactacaaggacgacgatgacaagtaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaaggtaggtaacaactccatactttttggttgtttattaatgtgaaatttctgctaaatgaaatacttttgtgtgtgtttgtggtagaagagaccacttcagttaaataaggaaatcaagagaggatcaatttaggttcgttttaaagagattaaaaaaaatcaagacataaaatctacccaagcaggatagaaatctccactgcaaagttccatgccaaagacatctggttatttttatttttaatggaagacttgaaggaatgataggtgattaataatgatcaaacagaagtctttaaatgttggaaagtatttacattaatctttgtatatatcattgggcattttagcacttgagagaaatagtttattaaagatataatcaatcatatgtaactgaacatttagaaaaattatatacaggtttgagtagcccttatctgaaacttttggggccagaagtgttttggattccagatttttccggattttggaatatttgcactgccaactagttaagcacccccaaatttgaaaattcgtttcctttgagtgtcatgtcaatgcccaaaaagtttcagatatttggatttgagatgctcaacctgtataaggattcagaaagttattctgattaatgattttaagattcagatatacagccgggtgcagtggctcatgcctgtaatccctgcacttagggaggctgaggcgggtggatgacctgaggttaggagttcaagaccagcctggccaacatggcgaaacccccatctctactaaaaataacaaaaattagctgggtgtggtggtgggtgtctataatcccagcaacttgggaggctgaggcaggagaatcacttgaacccaggagatggaggttgcagtgagccgagatcatgccattgcactccagcctgggccacaagagcaaaactctgtctcaaaaaaacaaaacaaaa

SEQ ID NO: 12 - DNA, 3079 bp, synthetic construct
  source 1..3079: mol_type = other DNA
ctttttttttgagacagagtcttgctctgtctcacaggctgaagtgcagtggtgcaatctcggctcactgcaacctccacctcccaggttcaagcgattctcctccctcagcctcccgagtagctgggaccacaggcatgcaccaccatccccagctaatttttgcattattagtagagttgggatttcttcaccgtgttggccaggctggtcttggactcctgacctcaagtgatccaactgcctcagcctctcaaagtgctaggattacagggatctatacttttcttttgagggaaaatgttggcaccgtttctagggcatattggccatttcagcttctcagtaaatatttgttaagtaattaaatgcacttgattctttattcttagccttttaacgcaatactcagaatagctgaagcaccaattaactgaaatggagatattataaagatagttatcttctccaagggaaaaaatcatcttcatggaaattaattacttttttacaaattgtgaatttgacccttaagagttttcttcctgatatttaaaattgaaaaaaaaattgttgacattaatatttcttctttccttttttttcttttcctttttttttttttttttgcaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggactacaaggacgacgatgacaagtaattaaaacagcctgtgggttgatcccacccacaggtaggtaacaactccatactttttggttgtttattaatgtgaaatttctgctaaatgaaatacttttgtgtgtgtttgtggtagaagagaccacttcagttaaataaggaaatcaagagaggatcaatttaggttcgttttaaagagattaaaaaaaatcaagacataaaatctacccaagcaggatagaaatctccactgcaaagttccatgccaaagacatctggttatttttatttttaatggaagacttgaaggaatgataggtgattaataatgatcaaacagaagtctttaaatgttggaaagtatttacattaatctttgtatatatcattgggcattttagcacttgagagaaatagtttattaaagatataatcaatcatatgtaactgaacatttagaaaaattatatacaggtttgagtagcccttatctgaaacttttggggccagaagtgttttggattccagatttttccggattttggaatatttgcactgccaactagttaagcacccccaaatttgaaaattcgtttcctttgagtgtcatgtcaatgcccaaaaagtttcagatatttggatttgagatgctcaacctgtataaggattcagaaagttattctgattaatgattttaagattcagatatacagccgggtgcagtggctcatgcctgtaatccctgcacttagggaggctgaggcgggtggatgacctgaggttaggagttcaagaccagcctggccaacatggcgaaacccccatctctactaaaaataacaaaaattagctgggtgtggtggtgggtgtctataatcccagcaacttgggaggctgaggcaggagaatcacttgaacccaggagatggaggttgcagtgagccgagatcatgccattgcactccagcctgggccacaagagcaaaactctgtctcaaaaaaacaaaacaaaa

SEQ ID NO: 13 - DNA, 3079 bp, synthetic construct
  source 1..3079: mol_type = other DNA
ctttttttttgagacagagtcttgctctgtctcacaggctgaagtgcagtggtgcaatctcggctcactgcaacctccacctcccaggttcaagcgattctcctccctcagcctcccgagtagctgggaccacaggcatgcaccaccatccccagctaatttttgcattattagtagagttgggatttcttcaccgtgttggccaggctggtcttggactcctgacctcaagtgatccaactgcctcagcctctcaaagtgctaggattacagggatctatacttttcttttgagggaaaatgttggcaccgtttctagggcatattggccatttcagcttctcagtaaatatttgttaagtaattaaatgcacttgattctttattcttagccttttaacgcaatactcagaatagctgaagcaccaattaactgaaatggagatattataaagatagttatcttctccaagggaaaaaatcatcttcatggaaattaattacttttttacaaattgtgaatttgacccttaagagttttcttcctgatatttaaaattgaaaaaaaaattgttgacattaatatttcttctttccttttttttcttttcctttttttttttttttttgcaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggactacaaggacgacgatgacaagtaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggtaggtaacaactccatactttttggttgtttattaatgtgaaatttctgctaaatgaaatacttttgtgtgtgtttgtggtagaagagaccacttcagttaaataaggaaatcaagagaggatcaatttaggttcgttttaaagagattaaaaaaaatcaagacataaaatctacccaagcaggatagaaatctccactgcaaagttccatgccaaagacatctggttatttttatttttaatggaagacttgaaggaatgataggtgattaataatgatcaaacagaagtctttaaatgttggaaagtatttacattaatctttgtatatatcattgggcattttagcacttgagagaaatagtttattaaagatataatcaatcatatgtaactgaacatttagaaaaattatatacaggtttgagtagcccttatctgaaacttttggggccagaagtgttttggattccagatttttccggattttggaatatttgcactgccaactagttaagcacccccaaatttgaaaattcgtttcctttgagtgtcatgtcaatgcccaaaaagtttcagatatttggatttgagatgctcaacctgtataaggattcagaaagttattctgattaatgattttaagattcagatatacagccgggtgcagtggctcatgcctgtaatccctgcacttagggaggctgaggcgggtggatgacctgaggttaggagttcaagaccagcctggccaacatggcgaaacccccatctctactaaaaataacaaaaattagctgggtgtggtggtgggtgtctataatcccagcaacttgggaggctgaggcaggagaatcacttgaacccaggagatggaggttgcagtgagccgagatcatgccattgcactccagcctgggccacaagagcaaaactctgtctcaaaaaaacaaaacaaaa

SEQ ID NO: 14 - DNA, 2981 bp, synthetic construct
  source 1..2981: mol_type = other DNA
ctttttttttgagacagagtcttgctctgtctcacaggctgaagtgcagtggtgcaatctcggctcactgcaacctccacctcccaggttcaagcgattctcctccctcagcctcccgagtagctgggaccacaggcatgcaccaccatccccagctaatttttgcattattagtagagttgggatttcttcaccgtgttggccaggctggtcttggactcctgacctcaagtgatccaactgcctcagcctctcaaagtgctaggattacagggatctatacttttcttttgagggaaaatgttggcaccgtttctagggcatattggccatttcagcttctcagtaaatatttgttaagtaattaaatgcacttgattctttattcttagccttttaacgcaatactcagaatagctgaagcaccaattaactgaaatggagatattataaagatagttatcttctccaagggaaaaaatcatcttcatggaaattaattacttttttacaaattgtgaatttgacccttaagagttttcttcctgatatttaaaattgaaaaaaaaattgttgacattaatatttcttctttccttttttttcttttcctttttttttttttttttgcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggactacaaggacgacgatgacaagtaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggtaggtaacaactccatactttttggttgtttattaatgtgaaatttctgctaaatgaaatacttttgtgtgtgtttgtggtagaagagaccacttcagttaaataaggaaatcaagagaggatcaatttaggttcgttttaaagagattaaaaaaaatcaagacataaaatctacccaagcaggatagaaatctccactgcaaagttccatgccaaagacatctggttatttttatttttaatggaagacttgaaggaatgataggtgattaataatgatcaaacagaagtctttaaatgttggaaagtatttacattaatctttgtatatatcattgggcattttagcacttgagagaaatagtttattaaagatataatcaatcatatgtaactgaacatttagaaaaattatatacaggtttgagtagcccttatctgaaacttttggggccagaagtgttttggattccagatttttccggattttggaatatttgcactgccaactagttaagcacccccaaatttgaaaattcgtttcctttgagtgtcatgtcaatgcccaaaaagtttcagatatttggatttgagatgctcaacctgtataaggattcagaaagttattctgattaatgattttaagattcagatatacagccgggtgcagtggctcatgcctgtaatccctgcacttagggaggctgaggcgggtggatgacctgaggttaggagttcaagaccagcctggccaacatggcgaaacccccatctctactaaaaataacaaaaattagctgggtgtggtggtgggtgtctataatcccagcaacttgggaggctgaggcaggagaatcacttgaacccaggagatggaggttgcagtgagccgagatcatgccattgcactccagcctgggccacaagagcaaaactctgtctcaaaaaaacaaaacaaaa

SEQ ID NO: 15 - DNA, 3384 bp, synthetic construct
  source 1..3384: mol_type = other DNA
ctttttttttgagacagagtcttgctctgtctcacaggctgaagtgcagtggtgcaatctcggctcactgcaacctccacctcccaggttcaagcgattctcctccctcagcctcccgagtagctgggaccacaggcatgcaccaccatccccagctaatttttgcattattagtagagttgggatttcttcaccgtgttggccaggctggtcttggactcctgacctcaagtgatccaactgcctcagcctctcaaagtgctaggattacagggatctatacttttcttttgagggaaaatgttggcaccgtttctagggcatattggccatttcagcttctcagtaaatatttgttaagtaattaaatgcacttgattctttattcttagccttttaacgcaatactcagaatagctgaagcaccaattaactgaaatggagatattataaagatagttatcttctccaagggaaaaaatcatcttcatggaaattaattacttttttacaaattgtgaatttgacccttaagagttttcttcctgatatttaaaattgaaaaaaaaattgttgacattaatatttcttctttccttttttttcttttcctttttttttttttttttgcaggttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggactacaaggacgacgatgacaagtaattcttatgcccgtcaaggtcaaatagaagtgagcatattagcaaggctcagtactgaaaatgctgatgaatataactttgtacgagcttatgaatgctttcagcaccgtaaccatacttgtttagtctttgagatgctggaacaaaacttgtatgactttctgaaacaaaataaatttagtcccctgccactaaaagtgattcggcccattcttcaacaagtggccactgcactgaaaaaattgaaaagtcttggtttaattcatgctgatctcaagccagagaatattatgttggtggatcctgttcggcagccttacagggttaaagtaatagactttgggtcggccagtcatgtatcaaagactgtttgttcaacatatctacaatctcggtactacaggtaggtaacaactccatactttttggttgtttattaatgtgaaatttctgctaaatgaaatacttttgtgtgtgtttgtggtagaagagaccacttcagttaaataaggaaatcaagagaggatcaatttaggttcgttttaaagagattaaaaaaaatcaagacataaaatctacccaagcaggatagaaatctccactgcaaagttccatgccaaagacatctggttatttttatttttaatggaagacttgaaggaatgataggtgattaataatgatcaaacagaagtctttaaatgttggaaagtatttacattaatctttgtatatatcattgggcattttagcacttgagagaaatagtttattaaagatataatcaatcatatgtaactgaacatttagaaaaattatatacaggtttgagtagcccttatctgaaacttttggggccagaagtgttttggattccagatttttccggattttggaatatttgcactgccaactagttaagcacccccaaatttgaaaattcgtttcctttgagtgtcatgtcaatgcccaaaaagtttcagatatttggatttgagatgctcaacctgtataaggattcagaaagttattctgattaatgattttaagattcagatatacagccgggtgcagtggctcatgcctgtaatccctgcacttagggaggctgaggcgggtggatgacctgaggttaggagttcaagaccagcctggccaacatggcgaaacccccatctctactaaaaataacaaaaattagctgggtgtggtggtgggtgtctataatcccagcaacttgggaggctgaggcaggagaatcacttgaacccaggagatggaggttgcagtgagccgagatcatgccattgcactccagcctgggccacaagagcaaaactctgtctcaaaaaaacaaaacaaaa

SEQ ID NO: 16 - DNA, 3381 bp, synthetic construct
  source 1..3381: mol_type = other DNA
ctttttttttgagacagagtcttgctctgtctcacaggctgaagtgcagtggtgcaatctcggctcactgcaacctccacctcccaggttcaagcgattctcctccctcagcctcccgagtagctgggaccacaggcatgcaccaccatccccagctaatttttgcattattagtagagttgggatttcttcaccgtgttggccaggctggtcttggactcctgacctcaagtgatccaactgcctcagcctctcaaagtgctaggattacagggatctatacttttcttttgagggaaaatgttggcaccgtttctagggcatattggccatttcagcttctcagtaaatatttgttaagtaattaaatgcacttgattctttattcttagccttttaacgcaatactcagaatagctgaagcaccaattaactgaaatggagatattataaagatagttatcttctccaagggaaaaaatcatcttcatggaaattaattacttttttacaaattgtgaatttgacccttaagagttttcttcctgatatttaaaattgaaaaaaaaattgttgacattaatatttcttctttccttttttttcttttcctttttttttttttttttgcaggtatggcctcacaagtcttggtctacccaccatatgtttatcaaactcagttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggactacaaggacgacgatgacaagtaaactgaaaatgctgatgaatataactttgtacgagcttatgaatgctttcagcaccgtaaccatacttgtttagtctttgagatgctggaacaaaacttgtatgactttctgaaacaaaataaatttagtcccctgccactaaaagtgattcggcccattcttcaacaagtggccactgcactgaaaaaattgaaaagtcttggtttaattcatgctgatctcaagccagagaatattatgttggtggatcctgttcggcagccttacagggttaaagtaatagactttgggtcggccagtcatgtatcaaagactgtttgttcaacatatctacaatctcggtactacaggtaggtaacaactccatactttttggttgtttattaatgtgaaatttctgctaaatgaaatacttttgtgtgtgtttgtggtagaagagaccacttcagttaaataaggaaatcaagagaggatcaatttaggttcgttttaaagagattaaaaaaaatcaagacataaaatctacccaagcaggatagaaatctccactgcaaagttccatgccaaagacatctggttatttttatttttaatggaagacttgaaggaatgataggtgattaataatgatcaaacagaagtctttaaatgttggaaagtatttacattaatctttgtatatatcattgggcattttagcacttgagagaaatagtttattaaagatataatcaatcatatgtaactgaacatttagaaaaattatatacaggtttgagtagcccttatctgaaacttttggggccagaagtgttttggattccagatttttccggattttggaatatttgcactgccaactagttaagcacccccaaatttgaaaattcgtttcctttgagtgtcatgtcaatgcccaaaaagtttcagatatttggatttgagatgctcaacctgtataaggattcagaaagttattctgattaatgattttaagattcagatatacagccgggtgcagtggctcatgcctgtaatccctgcacttagggaggctgaggcgggtggatgacctgaggttaggagttcaagaccagcctggccaacatggcgaaacccccatctctactaaaaataacaaaaattagctgggtgtggtggtgggtgtctataatcccagcaacttgggaggctgaggcaggagaatcacttgaacccaggagatggaggttgcagtgagccgagatcatgccattgcactccagcctgggccacaagagcaaaactctgtctcaaaaaaacaaaacaaaa

SEQ ID NO: 17 - DNA, 3381 bp, synthetic construct
  source 1..3381: mol_type = other DNA
ctttttttttgagacagagtcttgctctgtctcacaggctgaagtgcagtggtgcaatctcggctcactgcaacctccacctcccaggttcaagcgattctcctccctcagcctcccgagtagctgggaccacaggcatgcaccaccatccccagctaatttttgcattattagtagagttgggatttcttcaccgtgttggccaggctggtcttggactcctgacctcaagtgatccaactgcctcagcctctcaaagtgctaggattacagggatctatacttttcttttgagggaaaatgttggcaccgtttctagggcatattggccatttcagcttctcagtaaatatttgttaagtaattaaatgcacttgattctttattcttagccttttaacgcaatactcagaatagctgaagcaccaattaactgaaatggagatattataaagatagttatcttctccaagggaaaaaatcatcttcatggaaattaattacttttttacaaattgtgaatttgacccttaagagttttcttcctgatatttaaaattgaaaaaaaaattgttgacattaatatttcttctttccttttttttcttttcctttttttttttttttttgcaggtatggcctcacaagtcttggtctacccaccatatgtttatcaaactcagtcaagtgccttttgtagtgtgaagaaactcaaagtagagccaagcagttgttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggactacaaggacgacgatgacaagtaagcaccgtaaccatacttgtttagtctttgagatgctggaacaaaacttgtatgactttctgaaacaaaataaatttagtcccctgccactaaaagtgattcggcccattcttcaacaagtggccactgcactgaaaaaattgaaaagtcttggtttaattcatgctgatctcaagccagagaatattatgttggtggatcctgttcggcagccttacagggttaaagtaatagactttgggtcggccagtcatgtatcaaagactgtttgttcaacatatctacaatctcggtactacaggtaggtaacaactccatactttttggttgtttattaatgtgaaatttctgctaaatgaaatacttttgtgtgtgtttgtggtagaagagaccacttcagttaaataaggaaatcaagagaggatcaatttaggttcgttttaaagagattaaaaaaaatcaagacataaaatctacccaagcaggatagaaatctccactgcaaagttccatgccaaagacatctggttatttttatttttaatggaagacttgaaggaatgataggtgattaataatgatcaaacagaagtctttaaatgttggaaagtatttacattaatctttgtatatatcattgggcattttagcacttgagagaaatagtttattaaagatataatcaatcatatgtaactgaacatttagaaaaattatatacaggtttgagtagcccttatctgaaacttttggggccagaagtgttttggattccagatttttccggattttggaatatttgcactgccaactagttaagcacccccaaatttgaaaattcgtttcctttgagtgtcatgtcaatgcccaaaaagtttcagatatttggatttgagatgctcaacctgtataaggattcagaaagttattctgattaatgattttaagattcagatatacagccgggtgcagtggctcatgcctgtaatccctgcacttagggaggctgaggcgggtggatgacctgaggttaggagttcaagaccagcctggccaacatggcgaaacccccatctctactaaaaataacaaaaattagctgggtgtggtggtgggtgtctataatcccagcaacttgggaggctgaggcaggagaatcacttgaacccaggagatggaggttgcagtgagccgagatcatgccattgcactccagcctgggccacaagagcaaaactctgtctcaaaaaaacaaaacaaaa

SEQ ID NO: 18 - DNA, 3381 bp, synthetic construct
  source 1..3381: mol_type = other DNA
ctttttttttgagacagagtcttgctctgtctcacaggctgaagtgcagtggtgcaatctcggctcactgcaacctccacctcccaggttcaagcgattctcctccctcagcctcccgagtagctgggaccacaggcatgcaccaccatccccagctaatttttgcattattagtagagttgggatttcttcaccgtgttggccaggctggtcttggactcctgacctcaagtgatccaactgcctcagcctctcaaagtgctaggattacagggatctatacttttcttttgagggaaaatgttggcaccgtttctagggcatattggccatttcagcttctcagtaaatatttgttaagtaattaaatgcacttgattctttattcttagccttttaacgcaatactcagaatagctgaagcaccaattaactgaaatggagatattataaagatagttatcttctccaagggaaaaaatcatcttcatggaaattaattacttttttacaaattgtgaatttgacccttaagagttttcttcctgatatttaaaattgaaaaaaaaattgttgacattaatatttcttctttccttttttttcttttcctttttttttttttttttgcaggtatggcctcacaagtcttggtctacccaccatatgtttatcaaactcagtcaagtgccttttgtagtgtgaagaaactcaaagtagagccaagcagttgtgtattccaggaaagaaactatccacggacctatgtgaatggtagaaactttggaaattctcatcctcccactaagggtagtgcttttcagacaaagatattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggactacaaggacgacgatgacaagtaacggcccattcttcaacaagtggccactgcactgaaaaaattgaaaagtcttggtttaattcatgctgatctcaagccagagaatattatgttggtggatcctgttcggcagccttacagggttaaagtaatagactttgggtcggccagtcatgtatcaaagactgtttgttcaacatatctacaatctcggtactacaggtaggtaacaactccatactttttggttgtttattaatgtgaaatttctgctaaatgaaatacttttgtgtgtgtttgtggtagaagagaccacttcagttaaataaggaaatcaagagaggatcaatttaggttcgttttaaagagattaaaaaaaatcaagacataaaatctacccaagcaggatagaaatctccactgcaaagttccatgccaaagacatctggttatttttatttttaatggaagacttgaaggaatgataggtgattaataatgatcaaacagaagtctttaaatgttggaaagtatttacattaatctttgtatatatcattgggcattttagcacttgagagaaatagtttattaaagatataatcaatcatatgtaactgaacatttagaaaaattatatacaggtttgagtagcccttatctgaaacttttggggccagaagtgttttggattccagatttttccggattttggaatatttgcactgccaactagttaagcacccccaaatttgaaaattcgtttcctttgagtgtcatgtcaatgcccaaaaagtttcagatatttggatttgagatgctcaacctgtataaggattcagaaagttattctgattaatgattttaagattcagatatacagccgggtgcagtggctcatgcctgtaatccctgcacttagggaggctgaggcgggtggatgacctgaggttaggagttcaagaccagcctggccaacatggcgaaacccccatctctactaaaaataacaaaaattagctgggtgtggtggtgggtgtctataatcccagcaacttgggaggctgaggcaggagaatcacttgaacccaggagatggaggttgcagtgagccgagatcatgccattgcactccagcctgggccacaagagcaaaactctgtctcaaaaaaacaaaacaaaa

SEQ ID NO: 19 - DNA, 3384 bp, synthetic construct
  source 1..3384: mol_type = other DNA
ctttttttttgagacagagtcttgctctgtctcacaggctgaagtgcagtggtgcaatctcggctcactgcaacctccacctcccaggttcaagcgattctcctccctcagcctcccgagtagctgggaccacaggcatgcaccaccatccccagctaatttttgcattattagtagagttgggatttcttcaccgtgttggccaggctggtcttggactcctgacctcaagtgatccaactgcctcagcctctcaaagtgctaggattacagggatctatacttttcttttgagggaaaatgttggcaccgtttctagggcatattggccatttcagcttctcagtaaatatttgttaagtaattaaatgcacttgattctttattcttagccttttaacgcaatactcagaatagctgaagcaccaattaactgaaatggagatattataaagatagttatcttctccaagggaaaaaatcatcttcatggaaattaattacttttttacaaattgtgaatttgacccttaagagttttcttcctgatatttaaaattgaaaaaaaaattgttgacattaatatttcttctttccttttttttcttttcctttttttttttttttttgcaggtatggcctcacaagtcttggtctacccaccatatgtttatcaaactcagtcaagtgccttttgtagtgtgaagaaactcaaagtagagccaagcagttgtgtattccaggaaagaaactatccacggacctatgtgaatggtagaaactttggaaattctcatcctcccactaagggtagtgcttttcagacaaagataccatttaatagacctcgaggacacaacttttcattgcagacaagtgctgttgttttgaaaaacactgcaggtgctacaaaggtcatagcagctcaggcacagcaagctcacgtgcaggcacctcagattggggcgtggcgaaacagattgcatttcctagaaggcccccagcgatgtggattgaagcgcaagagtgaggattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggactacaaggacgacgatgacaagtaacaggtaggtaacaactccatactttttggttgtttattaatgtgaaatttctgctaaatgaaatacttttgtgtgtgtttgtggtagaagagaccacttcagttaaataaggaaatcaagagaggatcaatttaggttcgttttaaagagattaaaaaaaatcaagacataaaatctacccaagcaggatagaaatctccactgcaaagttccatgccaaagacatctggttatttttatttttaatggaagacttgaaggaatgataggtgattaataatgatcaaacagaagtctttaaatgttggaaagtatttacattaatctttgtatatatcattgggcattttagcacttgagagaaatagtttattaaagatataatcaatcatatgtaactgaacatttagaaaaattatatacaggtttgagtagcccttatctgaaacttttggggccagaagtgttttggattccagatttttccggattttggaatatttgcactgccaactagttaagcacccccaaatttgaaaattcgtttcctttgagtgtcatgtcaatgcccaaaaagtttcagatatttggatttgagatgctcaacctgtataaggattcagaaagttattctgattaatgattttaagattcagatatacagccgggtgcagtggctcatgcctgtaatccctgcacttagggaggctgaggcgggtggatgacctgaggttaggagttcaagaccagcctggccaacatggcgaaacccccatctctactaaaaataacaaaaattagctgggtgtggtggtgggtgtctataatcccagcaacttgggaggctgaggcaggagaatcacttgaacccaggagatggaggttgcagtgagccgagatcatgccattgcactccagcctgggccacaagagcaaaactctgtctcaaaaaaacaaaacaaaa

SEQ ID NO: 20 - DNA, 1083 bp, synthetic construct
  source 1..1083: mol_type = other DNA
atggcccagacacccgcattcaacaaacccaaagtagagttacacgtccacctggatggagccatcaagccagaaaccatcttatactttggcaagaagagaggcatcgccctcccggcagatacagtggaggagctgcgcaacattatcggcatggacaagcccctctcgctcccaggcttcctggccaagtttgactactacatgcctgtgattgcgggctgcagagaggccatcaagaggatcgcctacgagtttgtggagatgaaggcaaaggagggcgtggtctatgtggaagtgcgctatagcccacacctgctggccaattccaaggtggacccaatgccctggaaccagactgaaggggacgtcacccctgatgacgttgtggatcttgtgaaccagggcctgcaggagggagagcaagcatttggcatcaaggtccggtccattctgtgctgcatgcgccaccagcccagctggtcccttgaggtgttggagctgtgtaagaagtacaatcagaagaccgtggtggctatggacttggctggggatgagaccattgaaggaagtagcctcttcccaggccacgtggaagcctatgagggcgcagtaaagaatggcattcatcggaccgtccacgctggcgaggtgggctctcctgaggttgtgcgtgaggctgtggacatcctcaagacagagagggtgggacatggttatcacaccatcgaggatgaagctctctacaacagactactgaaagaaaacatgcactttgaggtctgcccctggtccagctacctcacaggcgcctgggatcccaaaacgacgcatgcggttgttcgcttcaagaatgataaggccaactactcactcaacacagacgaccccctcatcttcaagtccaccctagacactgactaccagatgaccaagaaagacatgggcttcactgaggaggagttcaagcgactgaacatcaacgcagcgaagtcaagcttcctcccagaggaagagaagaaggaacttctggaacggctctacagagaataccaagactacaaggacgacgatgacaagtag

SEQ ID NO: 21 - DNA, 909 bp, synthetic construct
  source 1..909: mol_type = other DNA
atgcggctgggcagtcctggactgctcttcctgctcttcagcagccttcgagctgatactcaggagaaggaagtcagagcgatggtaggcagcgacgtggagctcagctgcgcttgccctgaaggaagccgttttgacttaaatgatgtttacgtatattggcaaaccagtgagtcgaaaaccgtggtgacctaccacatcccacagaacagctccttggaaaacgtggacagccgctaccggaaccgagccctgatgtcaccggccggcatgctgcggggcgacttctccctgcgcttgttcaacgtcaccccccaggacgagcagaagtttcactgcctggtgttgagccaatccctgggattccaggaggttttgagcgttgaggttacactgcatgtggcagcaaacttcagcgtgcccgtcgtcagcgccccccacagcccctcccaggatgagctcaccttcacgtgtacatccataaacggctaccccaggcccaacgtgtactggatcaataagacggacaacagcctgctggaccaggctctgcagaatgacaccgtcttcttgaacatgcggggcttgtatgacgtggtcagcgtgctgaggatcgcacggacccccagcgtgaacattggctgctgcatagagaacgtgcttctgcagcagaacctgactgtcggcagccagacaggaaatgacatcggagagagagacaagatcacagagaatccagtcagtaccggcgagaaaaacgcggccacgtggagcatcctggctgtcctgtgcctgcttgtggtcgtggcggtggccataggctgggtgtgcagggaccgatgcctccaacacagctatgcaggtgcctgggctgtgagtccggagacagagctcactggccacgtttga

SEQ ID NO: 22 - DNA, 936 bp, synthetic construct
  source 1..936: mol_type = other DNA
atggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaa